(19) United States
(12) Patent Application Publication
(10) Pub. No.: US 2024/0390677 A1
(43) Pub. Date: Nov. 28, 2024

(54) MULTIFACTORIAL QUANTITATION OF NEUROSTIMULATION TREATMENTS FOR AUTONOMIC DISORDERS

(71) Applicant: Boston Scientific Neuromodulation Corporation, Valencia, CA (US)

(72) Inventors: Ismael Huertas Fernandez, Sevilla (ES); QUE T. Doan, West Hills, CA (US); Koenraad Meuwissen, 's-Hertohenbosch (NL)

(21) Appl. No.: 18/655,695

(22) Filed: May 6, 2024

Related U.S. Application Data
(60) Provisional application No. 63/468,702, filed on May 24, 2023.

(51) Int. Cl. A61N 1/36; A61N 1/372; G16H 10/20

(52) U.S. Cl. CPC A61N 1/36062; A61N 1/36132; A61N 1/36171; A61N 1/36175; A61N 1/36185; A61N 1/37247; G16H 10/20

(57) ABSTRACT

Systems and methods for planning neurostimulation programming for an autonomic condition of a patient are disclosed. A system may include a user interface device configured to provide a graphical user interface that receives patient data and outputs severity data corresponding to multiple characteristics of an autonomic condition of the patient. The system may also include a data processing system configured to: identify neural targets of the patient corresponding to respective leads and electrodes of a neurostimulation system; determine, based on the patient data, respective severity scores corresponding to the multiple characteristics of the autonomic condition; generate, using the respective severity scores, neurostimulation programming settings for use at the neural targets; and output programming data, based on the neurostimulation programming settings, for controlling neurostimulation with the neurostimulation system.

### CLAIM OF PRIORITY

This application claims the benefit of U.S. Provisional Application No. 63/468,702 filed on May 24, 2023, which is hereby incorporated by reference in its entirety.

### TECHNICAL FIELD

This document relates generally to medical devices, and more particularly, to systems, devices and methods for planning and controlling the modulation of autonomic nervous system functions via spinal cord stimulation.

### BACKGROUND

Neuromodulation systems have been developed to provide therapy for a variety of treatments such as Spinal Cord Stimulation (SCS) for controlling chronic pain syndromes. An implantable neuromodulation system may include an implantable neurostimulator, also referred to as an implantable pulse generator (IPG), which can electrically stimulate tissue or nerve centers to treat nervous or muscular disorders. In an example, an IPG can deliver electrical pulses to a specific region in a patient's spinal cord, such as particular spinal nerve roots or nerve bundles, to create an analgesic effect that masks pain sensation.

Neuromodulation such as SCS has been observed to have therapeutic effects in treating certain conditions or diseases including, for example, cardiac neuropathy, limb ischemia, impotence, endometriosis, and various conditions, dysfunctions, or symptoms associated with the autonomic nervous system (ANS) generally known as dysautonomia. Post-ganglionic axonal processes of motor neurons in the autonomic ganglia innervate organs and tissues throughout the body (eyes, salivary glands, heart, stomach, urinary bladder, blood vessels, etc.). For example, SCS at T1-T5 levels of the spinal cord or the associated spinal nerves can be used to treat cardiac neuropathy due to its therapeutic effect of stabilizing the ANS, reducing pathologic sympathetic tone, and preventing cardiac events such as ischemia. Another condition that can be treated or alleviated with SCS is visceral pain, which refers to pain originated from or related to internal organs or blood vessels, such as bladder pain, endometriosis, irritable bowel syndrome, and prostate pain. SCS can influence visceral function by modulation of the ANS through spinal segmental circuitry (e.g., sympathetic pre-ganglionic neurons), thereby alleviating visceral pain in certain internal organs.

### SUMMARY

Various embodiments discussed in this document may provide more efficient and effective techniques for the management and assessment of treatments for autonomic nervous system conditions, in connection with neuromodulation therapy delivered from SCS. For instance, in addition to assessing the effectiveness of neuromodulation to a pain treatment, the following provides approaches for adapting neuromodulation to treatments of multifactorial medical disorders (involving multiple body systems and organs) associated with the autonomous nervous system. Accordingly, the following provides a methodology to plan, recommend, and respond with treatments of various disorders or symptoms that affect multiple internal organs or tissues in a patient.

Example 1 is a system for planning neurostimulation of a patient, comprising: a user interface device configured to provide a graphical user interface, the graphical user interface to receive patient data and output severity data corresponding to multiple characteristics of an autonomic condition of the patient; and a data processing system communicatively coupled to the user interface device, the data processing system configured to: identify neural targets of the patient corresponding to respective leads and electrodes of a neurostimulation system; determine, based on the patient data, respective severity scores corresponding to the multiple characteristics of the autonomic condition; generate, using the respective severity scores, neurostimulation programming settings for use at the neural targets; and output programming data, based on the neurostimulation programming settings, for controlling neurostimulation with the neurostimulation system.

In Example 2, the subject matter of Example 1 optionally includes subject matter where the neural targets of the patient include spinal neural targets, and wherein the neurostimulation includes spinal cord stimulation (SCS) to be provided via the respective leads and electrodes to the spinal neural targets.

In Example 3, the subject matter of any one or more of Examples 1-2 optionally include subject matter where the data processing system is further configured to: communicate the severity data to the user interface device, wherein the user interface device is further configured to output a visualization of the respective severity scores that depicts relative severity for each of the multiple characteristics of the autonomic condition.

In Example 4, the subject matter of Example 3 optionally includes subject matter where the visualization of the respective severity scores comprises a circular graph that positions the multiple characteristics of the autonomic condition at respective locations in the circular graph, and wherein the circular graph includes an overlay area that maps the respective severity scores onto the respective locations in the circular graph.

In Example 5, the subject matter of Example 4 optionally includes subject matter where the circular graph is configured to receive user interaction to change the respective severity scores at the respective locations in the circular graph.

In Example 6, the subject matter of any one or more of Examples 1-5 optionally include subject matter where the respective severity scores are determined based on the patient data received in a symptom questionnaire, and wherein the symptom questionnaire provides a numerical measurement of effects of the autonomic condition in respective anatomical systems or organs that correspond to the multiple characteristics of the autonomic condition.

In Example 7, the subject matter of any one or more of Examples 1-6 optionally include subject matter where the neurostimulation programming settings are determined based on a mapping of the neural targets to a severity score value corresponding to each of the multiple characteristics of the autonomic condition.

In Example 8, the subject matter of Example 7 optionally includes subject matter where the mapping of the neural targets is based on a score computed from a likelihood of targeting a respective characteristic of the autonomic condition and a severity weight for the respective characteristic of the autonomic condition.

In Example 9, the subject matter of any one or more of Examples 1-8 optionally include subject matter where the user interface device is further configured to output an anatomical visualization including at least one visceral map or dermatomal map, the anatomical visualization corresponding to symptoms or treatment effects at the neural targets.

In Example 10, the subject matter of any one or more of Examples 1-9 optionally include subject matter where the user interface device is further configured to output a heat map corresponding to symptoms or treatment effects at the neural targets, wherein the heat map is overlaid on the neural targets on a visualization of a spinal cord for the patient.

In Example 11, the subject matter of any one or more of Examples 1-10 optionally include subject matter where the neurostimulation programming settings includes information for: one or more spatial targets, one or more frequencies, and one or more pulse-widths used for the neurostimulation; and wherein the neurostimulation programming settings are generated based on a configuration of the respective leads and electrodes of the neurostimulation system used for the patient, wherein the configuration provides specifications for: a type of implantable pulse generator, a type of lead, a number of leads, and a number of electrodes on respective leads.

In Example 12, the subject matter of any one or more of Examples 1-11 optionally include subject matter where the respective severity scores are represented in the graphical user interface within a defined range or groups of ranges for each of the multiple characteristics of the autonomic condition.

In Example 13, the subject matter of any one or more of Examples 1-12 optionally include programming circuitry configured to generate programming instructions for an electrostimulator of the neurostimulation system, the programming instructions corresponding to the neurostimulation programming settings to control stimulation of at respective neural targets of the patient via the respective leads and electrodes; and optionally, the programming instructions includes one or more stimulation parameters including: an electrode configuration; one or more stimulation pulse parameters including a pulse amplitude, a pulse width, or a stimulation frequency; a stimulation pulse waveform; an ON-OFF cycling scheme comprising an ON period for delivering stimulation pulses and a subsequent stimulation-free OFF period; or a charge per second (CPS) or a charge per hour (CPH) delivered to the a respective neural target.

Example 14 is a machine-readable medium including instructions, which when executed by a machine, cause the machine to perform the operations of the system of any of the Examples 1 to 13.

Example 15 is a method to perform the operations of the system of any of the Examples 1 to 13.

Example 16 is a data processing system for planning neurostimulation of a patient, comprising: at least one memory device configured to store patient data corresponding to multiple characteristics of an autonomic condition of the patient; at least one processor configured to: identify neural targets of the patient corresponding to respective leads and electrodes of a neurostimulation system; determine, based on the patient data, respective severity scores corresponding to the multiple characteristics of the autonomic condition; generate, using the respective severity scores, neurostimulation programming settings for use at the neural targets; and output programming data, based on the neurostimulation programming settings, for controlling neurostimulation with the neurostimulation system.

In Example 17, the subject matter of Example 16 optionally includes subject matter where the neural targets of the patient include spinal neural targets, and wherein the neurostimulation includes spinal cord stimulation (SCS) to be provided via the respective leads and electrodes to the spinal neural targets.

In Example 18, the subject matter of any one or more of Examples 16-17 optionally include subject matter where the data processing system is further configured to: communicate with a user interface device to output the respective severity scores in a graphical user interface, wherein the user interface device is configured to output a visualization of the respective severity scores that depicts relative severity for each of the multiple characteristics of the autonomic condition.

In Example 19, the subject matter of Example 18 optionally includes subject matter where the respective severity scores are represented in the graphical user interface within a defined range or groups of ranges for each of the multiple characteristics of the autonomic condition.

In Example 20, the subject matter of any one or more of Examples 18-19 optionally include subject matter where the visualization of the respective severity scores comprises a circular graph that positions the multiple characteristics of the autonomic condition at respective locations in the circular graph, wherein the circular graph includes an overlay area that maps the respective severity scores onto the respective locations in the circular graph, and wherein the circular graph is configured to receive user interaction to change the respective severity scores at the respective locations in the circular graph.

In Example 21, the subject matter of any one or more of Examples 16-20 optionally include subject matter where the data processing system is further configured to: communicate with a user interface device to represent the multiple characteristics of an autonomic condition of the patient, wherein the user interface device is further configured to output a graphical user interface including at least one of: an anatomical visualization including at least one visceral map or dermatomal map, the anatomical visualization corresponding to symptoms or treatment effects at the neural targets; or a heat map corresponding to symptoms or treatment effects at the neural targets, and wherein the heat map is overlaid on the neural targets on a visualization of a spinal cord for the patient.

In Example 22, the subject matter of any one or more of Examples 16-21 optionally include subject matter where the respective severity scores are determined based on the patient data received in a symptom questionnaire, and wherein the symptom questionnaire provides a numerical measurement of effects of the autonomic condition in respective anatomical systems or organs that correspond to the multiple characteristics of the autonomic condition.

In Example 23, the subject matter of any one or more of Examples 16-22 optionally include subject matter where the neurostimulation programming settings are determined based on a mapping of the neural targets to a severity score value corresponding to each of the multiple characteristics of the autonomic condition, and wherein the mapping of the neural targets is based on a score computed from a likelihood of targeting a respective characteristic of the autonomic condition and a severity weight for the respective characteristic of the autonomic condition.

In Example 24, the subject matter of any one or more of Examples 16-23 optionally include subject matter where the neurostimulation programming settings includes information for: one or more spatial targets, one or more frequencies, and one or more pulse-widths used for the neurostimulation; and wherein the neurostimulation programming settings are generated based on a configuration of the respective leads and electrodes of the neurostimulation system used for the patient, wherein the configuration provides specifications for: a type of implantable pulse generator, a type of lead, a number of leads, and a number of electrodes on respective leads.

In Example 25, the subject matter of any one or more of Examples 16-24 optionally include communication circuitry configured to provide programming instructions for an electrostimulator of the neurostimulation system, the programming instructions corresponding to the neurostimulation programming settings to control stimulation of at respective neural targets of the patient via the respective leads and electrodes; wherein the programming instructions includes one or more stimulation parameters including: an electrode configuration; one or more stimulation pulse parameters including a pulse amplitude, a pulse width, or a stimulation frequency; a stimulation pulse waveform; an ON-OFF cycling scheme comprising an ON period for delivering stimulation pulses and a subsequent stimulation-free OFF period; or a charge per second (CPS) or a charge per hour (CPH) delivered to a respective neural target.

Example 26 is a method for planning neurostimulation of a patient, comprising: receiving patient data corresponding to multiple characteristics of an autonomic condition of the patient; identifying neural targets of the patient corresponding to respective leads and electrodes of a neurostimulation system; determining, based on the patient data, respective severity scores corresponding to the multiple characteristics of the autonomic condition; generating, using the respective severity scores, neurostimulation programming settings for use at the neural targets; and outputting programming data, based on the neurostimulation programming settings, for controlling neurostimulation with the neurostimulation system.

In Example 27, the subject matter of Example 26 optionally includes subject matter where the neural targets of the patient include spinal neural targets, and wherein the neurostimulation includes spinal cord stimulation (SCS) to be provided via the respective leads and electrodes to the spinal neural targets.

In Example 28, the subject matter of any one or more of Examples 26-27 optionally include communicating with a user interface device to output the respective severity scores in a graphical user interface, wherein the user interface device is configured to output a visualization of the respective severity scores that depicts relative severity for each of the multiple characteristics of the autonomic condition.

In Example 29, the subject matter of Example 28 optionally includes subject matter where the respective severity scores are represented in the graphical user interface within a defined range or groups of ranges for each of the multiple characteristics of the autonomic condition.

In Example 30, the subject matter of any one or more of Examples 28-29 optionally include subject matter where the visualization of the respective severity scores comprises a circular graph that positions the multiple characteristics of the autonomic condition at respective locations in the circular graph, wherein the circular graph includes an overlay area that maps the respective severity scores onto the respective locations in the circular graph, and wherein the circular graph is configured to receive user interaction to change the respective severity scores at the respective locations in the circular graph.

In Example 31, the subject matter of any one or more of Examples 26-30 optionally include communicating with a user interface device to represent the multiple characteristics of an autonomic condition of the patient, wherein the user interface device is further configured to output a graphical user interface including at least one of: an anatomical visualization including at least one visceral map or dermatomal map, the anatomical visualization corresponding to symptoms or treatment effects at the neural targets; or a heat map corresponding to symptoms or treatment effects at the neural targets, and wherein the heat map is overlaid on the neural targets on a visualization of a spinal cord for the patient.

In Example 32, the subject matter of any one or more of Examples 26-31 optionally include subject matter where the respective severity scores are determined based on the patient data received in a symptom questionnaire, and wherein the symptom questionnaire provides a numerical measurement of effects of the autonomic condition in respective anatomical systems or organs that correspond to the multiple characteristics of the autonomic condition.

In Example 33, the subject matter of any one or more of Examples 26-32 optionally include subject matter where the neurostimulation programming settings are determined based on a mapping of the neural targets to a severity score value corresponding to each of the multiple characteristics of the autonomic condition, and wherein the mapping of the neural targets is based on a score computed from a likelihood of targeting a respective characteristic of the autonomic condition and a severity weight for the respective characteristic of the autonomic condition.

In Example 34, the subject matter of any one or more of Examples 26-33 optionally include subject matter where the neurostimulation programming settings includes information for: one or more spatial targets, one or more frequencies, and one or more pulse-widths used for the neurostimulation; and wherein the neurostimulation programming settings are generated based on a configuration of the respective leads and electrodes of the neurostimulation system used for the patient, wherein the configuration provides specifications for: a type of implantable pulse generator, a type of lead, a number of leads, and a number of electrodes on respective leads.

In Example 35, the subject matter of any one or more of Examples 26-34 optionally include communicating programming instructions for an electrostimulator of the neurostimulation system, the programming instructions corresponding to the neurostimulation programming settings to control stimulation of at respective neural targets of the patient via the respective leads and electrodes; wherein the programming instructions includes one or more stimulation parameters including: an electrode configuration; one or more stimulation pulse parameters including a pulse amplitude, a pulse width, or a stimulation frequency; a stimulation pulse waveform; an ON-OFF cycling scheme comprising an ON period for delivering stimulation pulses and a subsequent stimulation-free OFF period; or a charge per second (CPS) or a charge per hour (CPH) delivered to a respective neural target.

This Summary is an overview of some of the teachings of the present application and is not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIGS. 1A-1B illustrate a portion of a spinal cord.

FIG. 2 illustrates an embodiment of a neuromodulation system.

FIG. 3 illustrates an embodiment of a modulation device, such as may be implemented in the neuromodulation system of FIG. 2.

FIG. 4 illustrates an embodiment of a programming device, such as may be implemented as the programming device in the neuromodulation system of FIG. 2.

FIG. 5 illustrates an implantable neuromodulation system and portions of an environment in which system may be used.

FIG. 6 illustrates an embodiment of a Spinal Cord Stimulation (SCS) system.

FIG. 7 illustrates some features of the neuromodulation lead and a waveform generator.

FIG. 8 illustrates a partial view of both neuroanatomy and bony anatomy of the spinal column.

FIGS. 9A-9C illustrate a transverse top view, a coronal side view and an angled view, respectively, of a nerve root.

FIGS. 10A-10G illustrate various examples of lead placement on a spinal cord.

FIG. 10H illustrates fractionalization of anodic current delivered to the electrodes on an electrical neuromodulation lead.

FIG. 11 is a diagram illustrating portions of the autonomic nervous system and spinal column origins of spinal nerves provided to and innervating various organs and tissues.

FIG. 12 is a block diagram illustrating a neuromodulation system that can identify or provide neurostimulation to a neural target to avoid or alleviate autonomic symptoms associated with one or more medical conditions.

FIGS. 13A and 13B illustrate circle graphs used for representing severity values of multiple medical conditions.

FIG. 14 illustrates a scenario of condition and lead placement visualization for a particular patient neurostimulation treatment use case.

FIG. 15 illustrates data inputs and outputs that may be used with a treatment planning system for neurostimulation.

FIGS. 16A and 16B illustrate simplified examples of likelihood of targeting data values, severity data values, and calculations of such data values for use with a treatment planning system for neurostimulation.

FIG. 17 illustrates a flowchart of a processing method implemented by a system or device for planning neurostimulation of a patient for treatment of a patient autonomic condition.

FIG. 18 illustrates a block diagram of a system (e.g., a computing system) for performing analysis of autonomic nervous system condition data.

FIG. 19 illustrates a block diagram of a system (e.g., a computing system) to cause programming of an implantable electrical neurostimulation device.

FIG. 20 illustrates a block diagram of an example machine upon which any one or more of the techniques (e.g., methodologies) discussed herein may perform.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

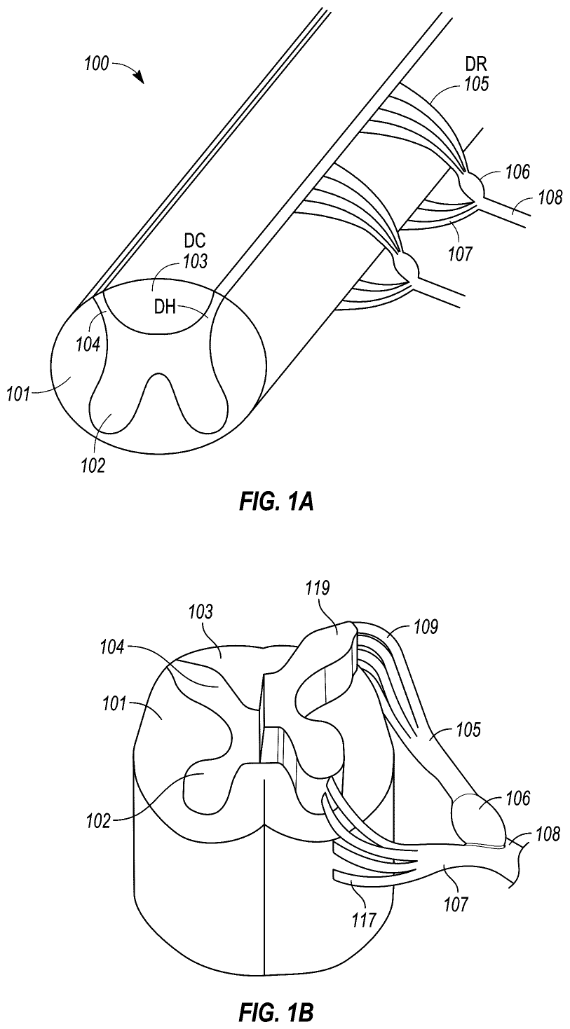

Various embodiments described herein involve treatment planning and device programming related to spinal cord stimulation (SCS) used for pain mitigation and a variety of other treatment effects. A brief description of the physiology of the spinal cord is provided herein to assist the reader. FIGS. 1A-1B illustrate, by way of example, a portion of a spinal cord 100 including white matter 101 and gray matter 102 of the spinal cord. The gray matter 102 includes cell bodies, synapse, dendrites, and axon terminals. Thus, synapses are located in the gray matter. White matter 101 includes myelinated axons that connect gray matter areas. A typical transverse section of the spinal cord includes a central “butterfly” shaped central area of gray matter 102 substantially surrounded by an ellipse-shaped outer area of white matter 101. The white matter of the dorsal column (DC) 103 includes mostly large myelinated axons that form afferent fibers that run in an axial direction. The dorsal portions of the “butterfly” shaped central area of gray matter are referred to as dorsal horns (DH) 104. In contrast to the DC fibers that run in an axial direction, DH fibers can be oriented in many directions, including perpendicular to the longitudinal axis of the spinal cord. FIGS. 1A-1B also illustrate spinal nerves, including a dorsal root (DR) 105, dorsal rootlets 109, dorsal root ganglion (DRG) 106, ventral root 107, and ventral rootlets 117. The dorsal root 105 mostly carries sensory signals into the spinal cord via a Dorsal Root Entry Zone (DREZ) 119 of the DH 104, and the ventral root 107 functions as an efferent motor root mostly carrying motor signals out of the spinal cord. The dorsal and ventral roots join to form mixed spinal nerve root 108.

SCS is often used to alleviate pain. A therapeutic goal for conventional SCS programming has been to maximize stimulation (i.e., recruitment) of the DC fibers that run in the white matter along the longitudinal axis of the spinal cord and minimal stimulation of other fibers that run perpendicular to the longitudinal axis of the spinal cord (dorsal root fibers, predominantly), as illustrated in FIGS. 1A-1B. The white matter of the DC includes mostly large myelinated axons that form afferent fibers. While the full mechanisms of pain relief are not well understood, it is believed that the perception of pain signals is inhibited via the gate control theory of pain, which suggests that enhanced activity of innocuous touch or pressure afferents via electrical stimulation creates interneuronal activity within the DH of the spinal cord that releases inhibitory neurotransmitters (Gamma-Aminobutyric Acid (GABA), glycine), which in turn, reduces the hypersensitivity of wide dynamic range (WDR) sensory neurons to noxious afferent input of pain signals traveling from the dorsal root (DR) neural fibers that innervate the pain region of the patient, as well as treating general WDR ectopy. Consequently, the large sensory afferents of the DC nerve fibers have been targeted for stimulation at an amplitude that provides pain relief.

An implantable neuromodulation system can include electrodes implanted adjacent, i.e., resting near, or upon the dura, to the dorsal column of the spinal cord of the patient and along a longitudinal axis of the spinal cord of the patient. In some examples, electrodes can be such placed to selectively or preferentially stimulate DR tissue over other neural tissue, such as but not limited to dorsal roots, dorsal rootlets, DRG, DREZ, or Lissauer's track. A lead or leads, including a plurality of electrodes, may be positioned to place the plurality of electrodes in proximity to a targeted nerve root. For example, the electrodes may be placed adjacent to the targeted nerve root, dorsal rootlets, or DREZ. The lead(s) may be placed using surgical approaches such as a lateral anterograde approach, a lateral retrograde approach, a sacral hiatus approach, or a transgrade approach. The lateral anterograde approach inserts the lead epidurally lower than the target, and then advances the lead in an anterograde direction (toward the head) until the lead is at the targeted nerve root. The lateral retrograde approach may be used to pass the lead closer to the DRG for selective root stimulation by inserting the lead epidurally above the target, and then advancing the lead in a retrograde direction (away from the head) to the targeted nerve root. The sacral hiatus approach introduces the introducer needle through the sacral hiatus into the epidural space and advanced in an anterograde direction (toward the head) to the targeted nerve root. Upon reaching the targeted nerve, the lead may be steered through the foramen to position extraforaminal, foraminal, and intraspinal electrodes along the targeted nerve root. The transgrade approach accesses the contralateral interlaminar space and steers the lead out of the opposite foramen to position extraforaminal, foraminal, and intraspinal electrodes along the targeted nerve root.

Stimulation of DR tissue may be useful to treat focal pain as it may provide the desired coverage for the pain without the stimulation spill over that can cause undesired effects in other areas of the body. Stimulation of DR tissue may be useful for delivering sub-perception therapy, which avoids the paresthesia that accompanies conventional SCS therapy when the large sensory DC nerve fibers are activated. Patients sometimes report these sensations to be unwanted. Sub-perception therapy may effectively treat pain without the patient sensing the delivery of the modulation field (e.g. paresthesia). Selective modulation of DR tissue, for either sub-perception therapy or to treat focal pain, may be delivered at higher frequencies (e.g. over 1,500 Hz such as frequencies within a range of 2 kHz to 20 kHz) or may be delivered at lower frequencies (e.g. at or less than 1,500 Hz such as frequencies at or less than 1,200 Hz, frequencies at or less than 1,000 Hz, frequencies at or less than 500 Hz, frequencies at or less than 350 Hz, or at or less than 130 Hz. The selective modulation may be delivered at low frequencies (e.g. as low as 2 Hz) or may be delivered even without pulses (e.g. 0 Hz). By way of example and not limitation, the selective modulation may be delivered within a frequency range selected from the following frequency ranges: 2 Hz to 1,200 Hz; 2 Hz to 1,000 Hz, 2 Hz to 500 Hz; 2 Hz to 350 Hz; or 2 Hz to 130 Hz. Systems may be developed to raise the lower end of any these ranges from 2 Hz to other frequencies such as, by way of example and not limitation, 10 Hz, 20 Hz, 50 Hz or 100 Hz. By way of example and not limitation, it is further noted that the selective modulation may be delivered with a duty cycle, in which stimulation (e.g. a train of pulses) is delivered during a Stimulation ON portion of the duty cycle, and is not delivered during a Stimulation OFF portion of the duty cycle. By way of example and not limitation, the duty cycle may be about 10%±5%, 20%±5%, 30%±5%, 40%±5%, 50%±5% or 60%±5%. For example, a burst of pulses for 10 ms during a Stimulation ON portion followed by 15 ms without pulses corresponds to a 40% duty cycle. Some waveforms may combine lower frequency pulses and higher frequency pulses into a more complex waveform (e.g. bursts of higher frequency pulses interleaved between one or more pulses delivered at a lower frequency. The waveform may have a regular pattern of pulses that repeats at regular intervals between pulses or regular intervals between burst of pulses. The waveform may have an irregular pattern of pulse that includes different intervals between pulses and/or different intervals between burst of pulses. The waveform may comprise rectilinear pulses, or may include other morphological shapes that are not rectilinear.

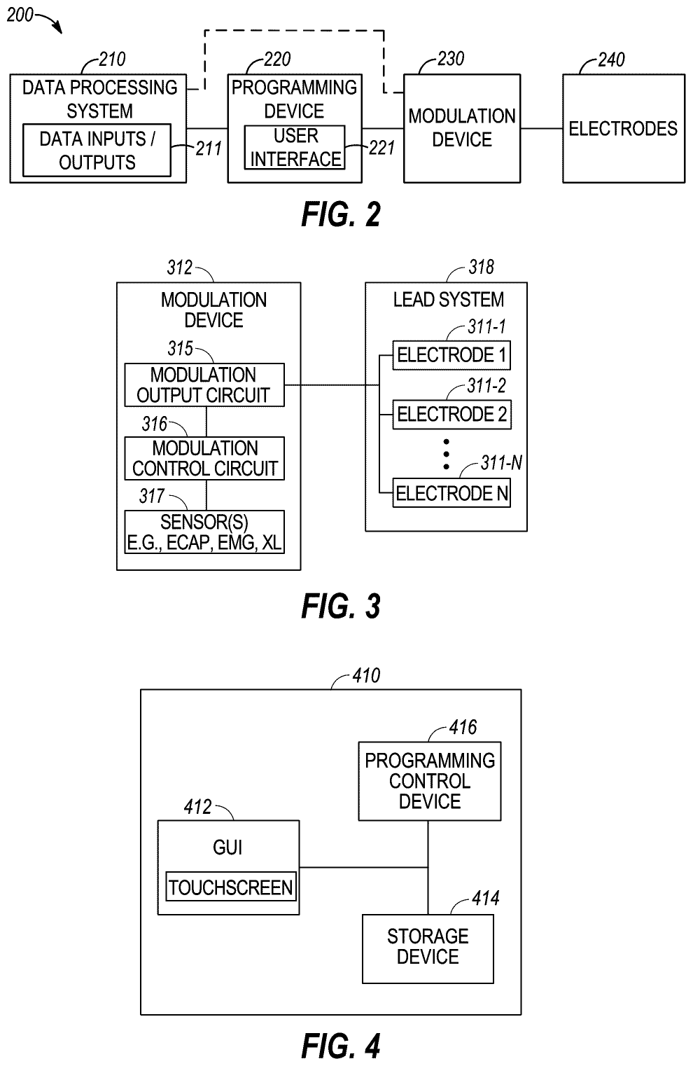

FIG. 2 illustrates an embodiment of a neuromodulation system 200. The illustrated system 200 includes electrodes 240, a modulation device 230, a programming device 220, and a data processing system 210. The electrodes 240 are configured to be placed on or near one or more neural targets in a patient, such as one or more dorsal nerve roots. The modulation device 230 is configured to be electrically connected to electrodes 240 and deliver neuromodulation energy, such as in the form of electrical pulses or other waveform shape, to the one or more neural targets though electrodes 240. The modulation device 230 may be an implantable device or an external device with leads percutaneously inserted to be positioned to stimulate a dorsal root. The delivery of the neuromodulation is controlled by using a plurality of modulation parameters, such as modulation parameters specifying the electrical pulses and a selection of electrode(s) to function as anode(s) and a selection of electrode(s) to function as cathode(s) through which each of the electrical pulses is delivered. The modulation parameter may also include the fractional distribution of energy (e.g. current) provided across the anodic electrode(s) and cathodic electrode(s). In various embodiments, at least some parameters of the plurality of modulation parameters are programmable by a user, such as a physician or other caregiver. The programming device 220 provides the user with accessibility to the user-programmable parameters. In various embodiments, the programming device 220 is configured to be communicatively coupled to modulation device via a wired or wireless link. In various embodiments, the programming device 220 includes a user interface 221 such as a graphical user interface (GUI) that allows the user to set and/or adjust values of the user-programmable modulation parameters. In some embodiments, data is provided to a data processing system 210 that provides various data inputs and outputs 211 to assist the user with the operation, configuration, programming, maintenance, or improvement of the modulation device 230. For example, in addition to planning of SCS stimulation for pain treatment, the data processing system 210 may assist with the planning of SCS stimulation for autonomic symptoms or side effects, including to treat or alleviate autonomic nervous system disorders as discussed in more detail below.

FIG. 3 illustrates an embodiment of a neuromodulation device 312, such as may be implemented in the neuromodulation system 200 of FIG. 2. The illustrated embodiment of the neuromodulation device 312 includes a neuromodulation output circuit 315 and a neuromodulation control circuit 316. Those of ordinary skill in the art will understand that the neuromodulation device 312 may include additional components such as sensing circuitry and sensors 317 for patient monitoring and/or feedback control of the therapy, telemetry circuitry and power. The neuromodulation output circuit 315 produces and delivers neuromodulation pulses. The neuromodulation control circuit 316 controls the delivery of the neuromodulation pulses using the plurality of neuromodulation parameters. The combination of the neuromodulation output circuit 315 and neuromodulation control circuit 316 may collectively be referred to as a pulse generator. The lead system 318 includes one or more leads each configured to be electrically connected to neuromodulation device 312 and a plurality of electrodes 311-1 to 311-N (where N≥2) distributed in an electrode arrangement using the one or more leads. Each lead may have an electrode array consisting of two or more electrodes, which also may be referred to as contacts. Multiple leads may provide multiple electrode arrays to provide the electrode arrangement. Each electrode is a single electrically conductive contact providing for an electrical interface between neuromodulation output circuit 315 and tissue of the patient. The neuromodulation pulses are each delivered from the neuromodulation output circuit 315 through a set of electrodes selected from the electrodes 311-1 to 311-N. The number of leads and the number of electrodes on each lead may depend on, for example, the distribution of target(s) of the neuromodulation and the need for controlling the distribution of electric field at each target. In one embodiment, by way of example and not limitation, the lead system includes two leads each having eight electrodes. Some embodiments may use a lead system that includes one or more leads of the same or different types such as percutaneous leads, linear paddles, multiple-column paddles, or directional leads, among others.

The neuromodulation system may be configured to modulate spinal target tissue or other neural tissue. The configuration of electrodes used to deliver electrical pulses to the targeted tissue constitutes an electrode configuration, with the electrodes capable of being selectively programmed to act as anodes (positive), cathodes (negative), or left off (zero). In other words, an electrode configuration represents the polarity being positive, negative, or zero. An electrical waveform may be controlled or varied for delivery using electrode configuration(s). The electrical waveforms may be analog or digital signals. In some embodiments, the electrical waveform includes pulses. The pulses may be delivered in a regular, repeating pattern, or may be delivered using complex patterns of pulses that appear to be irregular. Other parameters that may be controlled or varied include the amplitude, pulse width, and rate (or frequency) of the electrical pulses. Each electrode configuration, along with the electrical pulse parameters, can be referred to as a “neuromodulation parameter set.” Each set of neuromodulation parameters, including fractionalized current distribution to the electrodes (as percentage cathodic current, percentage anodic current, or off), may be stored and combined into a neuromodulation program that can then be used to modulate multiple regions within the patient.

The number of electrodes available combined with the ability to generate a variety of complex electrical pulses, presents a large selection of neuromodulation parameter sets to the clinician or patient. For example, if the neuromodulation system to be programmed has sixteen electrodes, millions of neuromodulation parameter sets may be available for programming into the neuromodulation system. Furthermore, as an example, SCS systems may have 32 electrodes (plus an additional electrode of the “can” or enclosure of the device) which exponentially increases the number of neuromodulation parameters sets available for programming. To facilitate such selection, the clinician generally programs the neuromodulation parameters sets through a computerized programming system to allow the optimum neuromodulation parameters to be determined based on patient feedback or other means and to subsequently program the desired neuromodulation parameter sets.

Patient paresthesia perception may be used to program SCS therapy, such as by selecting or determining an appropriate neuromodulation parameter set. The paresthesia induced by neuromodulation and perceived by the patient may be located in approximately the same places of the patient body where pain is sensed and thus the target site of treatment. Conventionally, when leads are implanted within the patient, an operating room (OR) mapping procedure may be performed to apply neuromodulation to test placement of the leads and/or electrodes, thereby assuring that the leads and/or electrodes are implanted in effective locations within the patient.

Once the leads are correctly positioned, a fitting procedure, which may be referred to as a navigation session, may be performed to program the external control device, and if applicable the neuromodulation device, with a set of neuromodulation parameters that best addresses the painful site. Thus, the navigation session may be used to pinpoint the volume of activation (VOA) or areas correlating to the pain. The procedure may be implemented to target the tissue during implantation, or after implantation should the leads gradually or unexpectedly move that would otherwise relocate the neuromodulation energy away from the target site. By reprogramming the neuromodulation device (typically by independently varying the neuromodulation energy on the electrodes), the VOA can often be moved back to the effective pain site without having to re-operate on the patient in order to reposition the lead and its electrode array. In addition to the information of dermatomal coverage such as correspondence between body sites of pain and body sites of induced paresthesia, one or more of patient information such as feedback on the induced paresthesia or patient perception thresholds may be used to optimize the target neuromodulation field. This may not only improve the neuromodulation precision and thus better therapeutic outcome, but may also save a system operator's time and ease the burden of programming a neuromodulation system.

FIG. 4 illustrates an embodiment of a programming device 410, such as may be implemented as the programming device 220 in the neuromodulation system of FIG. 2. The programming device 410 includes a storage device 414, a programming control device 416 (e.g., circuitry), and a GUI 412. The programming control device 416 generates the plurality of modulation parameters that controls the delivery of the neuromodulation pulses according to the pattern of the neuromodulation pulses. In various embodiments, the GUI 412 includes any type of presentation device, such as interactive or non-interactive screens, and any type of user input devices that allow the user to program the modulation parameters, such as touchscreen, keyboard, keypad, touchpad, trackball, joystick, and mouse. The storage device 414 may store, among other things, modulation parameters to be programmed into the modulation device. The modulation parameters may be organized into one or more sets of modulation parameters. The programming device 410 may transmit the plurality of modulation parameters to the modulation device. In some embodiments, the programming device 410 may transmit power to the modulation device. The programming control device 416 may generate the plurality of modulation parameters. In various embodiments, the programming control device 416 may check values of the plurality of modulation parameters against safety rules to limit these values within constraints of the safety rules.

In various embodiments, circuits of neuromodulation, including its various embodiments discussed in this document, may be implemented using a combination of hardware, software, and firmware. For example, the circuitry to implement the GUI, modulation control, and programming, including their various embodiments discussed in this document, may be implemented using application-specific circuit or circuitry constructed to perform one or more particular functions or a general-purpose circuit or circuitry programmed to perform such function(s). Such a general-purpose circuit includes, but is not limited to, a microprocessor or a portion thereof, a microcontroller or portions thereof, and a programmable logic circuit or a portion thereof.

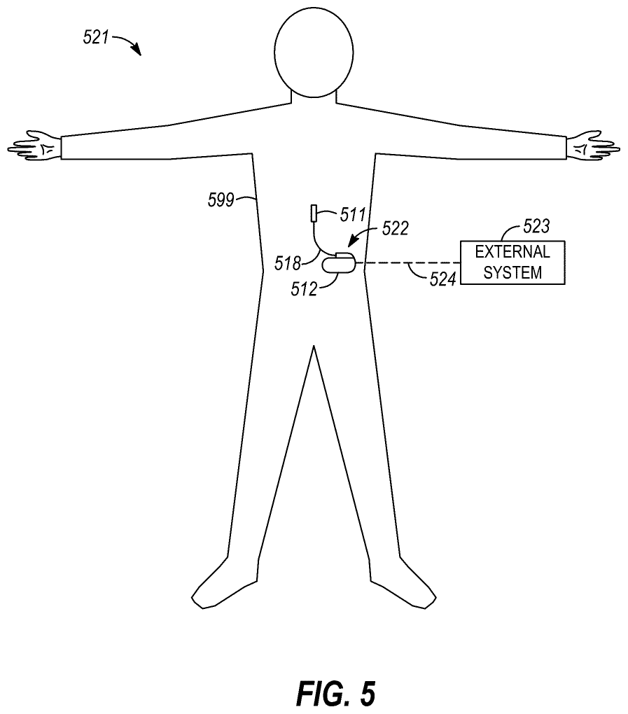

FIG. 5 illustrates, by way of example, an implantable neuromodulation system 521 and portions of an environment in which system may be used. The system is illustrated for implantation near the spinal cord of a patient 599. The system 521 includes an implantable medical device 522, an external system 523, and a telemetry link 524 providing for wireless communication between implantable medical device 522 and external system 523. The implantable system is illustrated as being implanted in the patient's body. The implantable medical device 522 includes an implantable modulation device (also referred to as an implantable pulse generator, or IPG) 512, a lead system 518, and electrodes 511. The lead system 518 includes one or more leads each configured to be electrically connected to the modulation device 512 and a plurality of electrodes 511 distributed in the one or more leads. In various embodiments, the external system 523 includes one or more external (non-implantable) devices each allowing a user (e.g. a clinician or other caregiver and/or the patient) to communicate with the implantable medical device 522. In some embodiments, the external system 523 includes a programming device intended for a clinician or other caregiver to initialize and adjust settings for the implantable medical device 522 and a remote control device intended for use by the patient. For example, the remote control device may allow the patient to turn a therapy on and off and/or adjust certain patient-programmable parameters of the plurality of modulation parameters. The external system 523 may include other local or remote servers or computer systems accessible through a variety of network(s).

The neuromodulation lead(s) of the lead system 518 may be placed proximate to (e.g. such as resting near, or upon the dura, adjacent to) the dorsal root tissue to be stimulated. Due to the lack of space near the location of the implanted neuromodulation lead(s), the implantable modulation device 512 may be implanted in a surgically-made pocket either in the abdomen or above the buttocks, or may be implanted in other locations of the patient's body. The lead extension(s) may be used to facilitate the implantation of the implantable modulation device 512 away from the exit point of the neuromodulation lead(s).

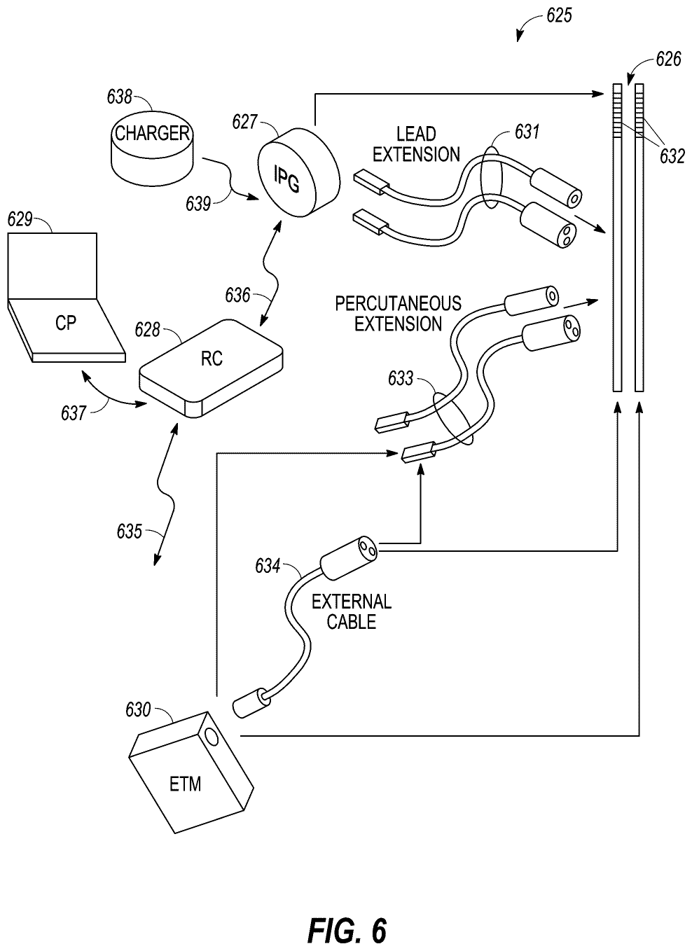

FIG. 6 illustrates, by way of example, an embodiment of a SCS system, which also may be referred to as a Spinal Cord Modulation system. The SCS system 625 may generally include a one or more (illustrated as two) of implantable neuromodulation leads 626, an implantable pulse generator (IPG) 627, an external remote controller RC 628, a clinician's programmer (CP) 629, and an external trial modulator (ETM) 630. The IPG 627 may be physically connected via one or more percutaneous lead extensions 631 to the neuromodulation lead(s) 626, which carry a plurality of electrodes 632. The electrodes, when implanted in a patient, form an electrode arrangement. As illustrated, the neuromodulation leads 626 may be percutaneous leads with the electrodes arranged in-line along the neuromodulation leads. Any suitable number of neuromodulation leads can be provided, including only one, as long as the number of electrodes is greater than two (including the IPG case function as a case electrode) to allow for lateral steering of the current. Alternatively, a surgical paddle lead can be used in place of one or more of the percutaneous leads. The IPG 627 includes pulse generation circuitry that delivers electrical modulation energy in the form of a pulsed electrical waveform (i.e., a temporal series of electrical pulses) to the electrodes in accordance with a set of modulation parameters.

The ETM 630 may also be physically connected via the percutaneous lead extensions 633 and external cable 634 to the neuromodulation lead(s) 626. The ETM 630 may have similar pulse generation circuitry as the IPG 627 to deliver electrical modulation energy to the electrodes accordance with a set of modulation parameters. The ETM 630 is a non-implantable device that may be used on a trial basis after the neuromodulation leads 626 have been implanted and prior to implantation of the IPG 627, to test the responsiveness of the modulation that is to be provided. Functions described herein with respect to the IPG 627 can likewise be performed with respect to the ETM 630.

The RC 628 may be used to telemetrically control the ETM 630 via a bi-directional RF communications link 635. The RC 628 may be used to telemetrically control the IPG 627 via a bi-directional RF communications link 636. Such control allows the IPG 627 to be turned on or off and to be programmed with different modulation parameter sets. The IPG 627 may also be operated to modify the programmed modulation parameters to actively control the characteristics of the electrical modulation energy output by the IPG 627. A clinician may use the CP 629 to program modulation parameters into the IPG 627 and ETM 630 in the operating room and in follow-up sessions.

The CP 629 may indirectly communicate with the IPG 627 or ETM 630, through the RC 628, via an IR communications link 637 or other link. The CP 629 may directly communicate with the IPG 627 or ETM 630 via an RF communications link or other link (not shown). The clinician detailed modulation parameters provided by the CP 629 may also be used to program the RC 628, so that the modulation parameters can be subsequently modified by operation of the RC 628 in a stand-alone mode (i.e., without the assistance of the CP 629). Various devices may function as the CP 629. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 629. Alternatively, such programming methodologies can be performed using firmware or hardware. In any event, the CP 629 may actively control the characteristics of the electrical modulation generated by the IPG 627 to allow the desired parameters to be determined based on patient feedback or other feedback and for subsequently programming the IPG 627 with the desired modulation parameters. To allow the user to perform these functions, the CP 629 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g. CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The display screen(s) may be used to suggest the electrode(s) for use to stimulate a targeted dorsal root. The external device(s) (e.g. CP and/or RC) may be configured to communicate with other device(s), including local device(s) and/or remote device(s). For example, wired and/or wireless communication may be used to communicate between or among the devices.

An external charger 638 may be a portable device used to transcutaneously charge the IPG via a wireless link such as an inductive link 639. Once the IPG has been programmed, and its power source has been charged by the external charger or otherwise replenished, the IPG may function as programmed without the RC or CP being present.

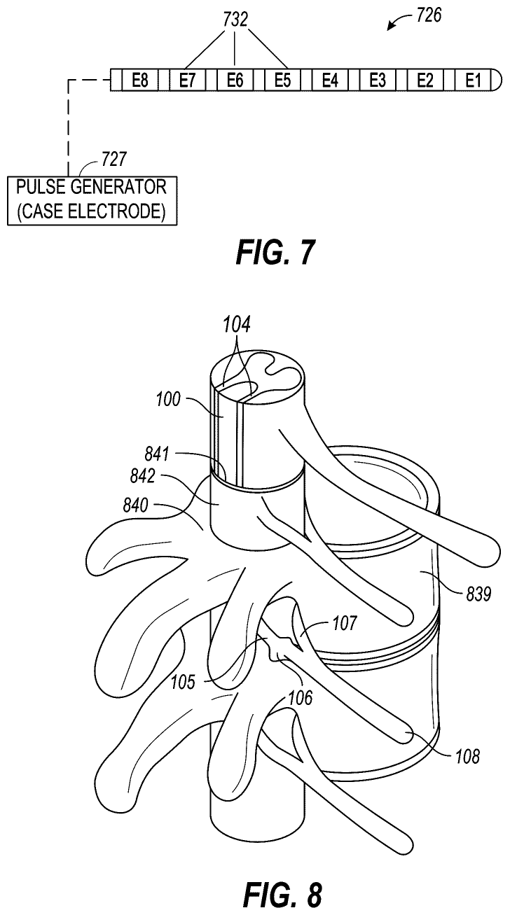

FIG. 7 illustrates, by way of example, some features of the neuromodulation leads 726 and a pulse generator 727. The pulse generator 727 may be an implantable device (IPG) or may be an external device such as may be used to test the electrodes during an implantation procedure. In the illustrated example, the neuromodulation lead has eight electrodes 732 (labeled E1-E8). The actual number and shape of leads and electrodes may vary for the intended application. An implantable pulse generator (IPG) may include an outer case for housing the electronic and other components. The outer case may be composed of an electrically conductive, biocompatible material, such as titanium, that forms a hermetically-sealed compartment wherein the internal electronics are protected from the body tissue and fluids. In some cases, the outer case may serve as an electrode (e.g. case electrode). The IPG may include electronic components, such as a controller/processor (e.g., a microcontroller), memory, a battery, telemetry circuitry, monitoring circuitry, modulation output circuitry, and other suitable components known to those skilled in the art. The microcontroller executes a suitable program stored in memory, for directing and controlling the neuromodulation performed by IPG. Electrical modulation energy is provided to the electrodes in accordance with a set of modulation parameters programmed into the pulse generator. The electrical modulation energy may be in the form of a pulsed electrical waveform. Such modulation parameters may comprise electrode combinations, which define the electrodes that are activated as anodes (positive), cathodes (negative), and turned off (zero), percentage of modulation energy assigned to each electrode (fractionalized electrode configurations), and electrical pulse parameters, which define the pulse amplitude (which may be measured in milliamps or volts depending on whether the pulse generator supplies constant current or constant voltage to the electrode array), pulse width (which may be measured in microseconds), pulse rate (which may be measured in pulses per second), and burst rate (which may be measured as the modulation on duration X and modulation off duration Y). Electrodes that are selected to transmit or receive electrical energy are referred to herein as “activated,” while electrodes that are not selected to transmit or receive electrical energy are referred to herein as “non-activated.”

Electrical modulation occurs between or among a plurality of activated electrodes, one of which may be the IPG case. The system may be capable of transmitting modulation energy to the tissue in a monopolar or multipolar (e.g., bipolar, tripolar, etc.) fashion. Monopolar modulation occurs when a selected one of the lead electrodes is activated along with the case of the IPG, so that modulation energy is transmitted between the selected electrode and case. Any of the electrodes E1-E8 and the case electrode may be assigned to up to k possible groups or timing “channels.” In one embodiment, k may equal four. The timing channel identifies which electrodes are selected to synchronously source or sink current to create an electric field in the tissue to be stimulated. Amplitudes and polarities of electrodes on a channel may vary. In particular, the electrodes can be selected to be positive (anode, sourcing current), negative (cathode, sinking current), or off (no current) polarity in any of the k timing channels. The IPG may be operated in a mode to deliver electrical modulation energy that is therapeutically effective and causes the patient to perceive delivery of the energy (e.g. therapeutically effective to relieve pain with perceived paresthesia), and may be operated in a sub-perception mode to deliver electrical modulation energy that is therapeutically effective and does not cause the patient to perceive delivery of the energy (e.g. therapeutically effective to relieve pain without perceived paresthesia).

The IPG may be configured to individually control the magnitude of electrical current flowing through each of the electrodes. For example, a current generator may be configured to selectively generate individual current-regulated amplitudes from independent current sources for each electrode. In some embodiments, the pulse generator may have voltage regulated outputs. While individually programmable electrode amplitudes are desirable to achieve fine control of the shape and size of the resulting modulation field, a single output source switched across electrodes may also be used, although with less fine control in programming. Neuromodulators may be designed with mixed current and voltage regulated devices.

FIG. 8 illustrates, for the convenience of the reader, a partial view of both neuroanatomy and bony anatomy of the spinal column. The neuroanatomy includes the spinal cord 100 such as was illustrated in FIGS. 1A-1B. The neuroanatomy also includes the dorsal horn (DH) 104, the dorsal root 105, the DRG 106, the ventral root 107, and the mixed spinal nerve root 108. The bony anatomy refers to the vertebrae that includes a vertebral body 839 and a bony ring 840 attached to the vertebral body 839. The stacked vertebrae provide a vertebral canal that protects the spinal cord 100. Nerve roots branch off and exit the spine on both sides through spaces (‘intervertebral foramen”) between the vertebra. The spinal cord is surrounded by dura matter 841, which holds spinal fluid that surrounds the spinal cord 100. The space between the walls and the dura matter of the vertebral canal is referred to as epidural space 842.

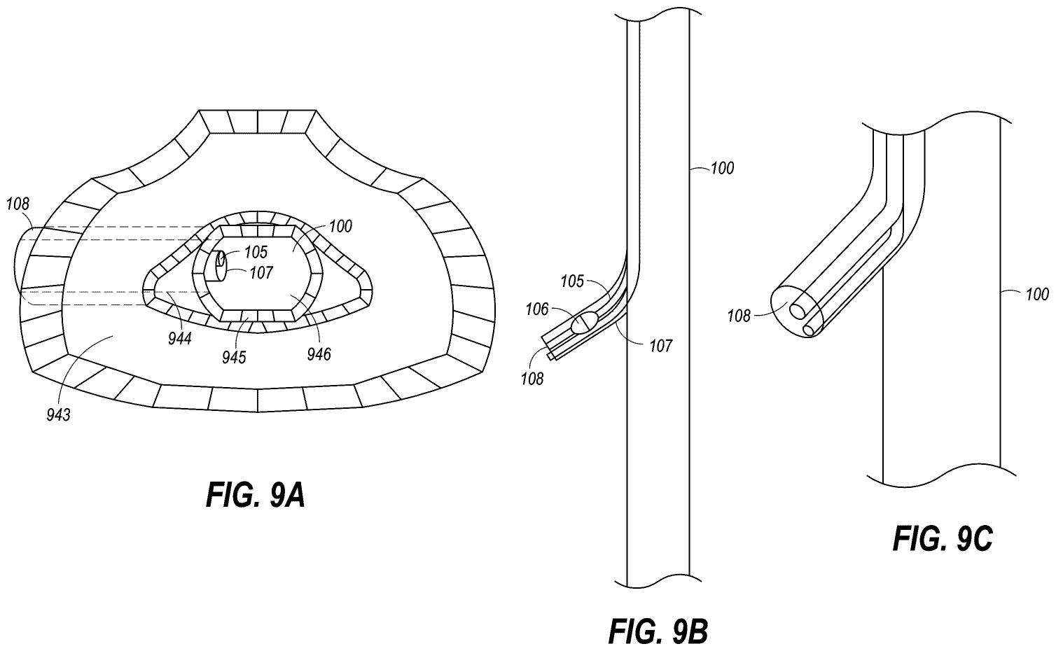

FIGS. 9A-9C illustrate a transverse top view, a coronal side view and an angled view, respectively, of the spinal cord 100, the dorsal root 105, the DRG 106, the ventral root 107 and the mixed spinal nerve root 108. FIG. 9A also illustrates bone 943, fat 944, dura 945 and cerebrospinal fluid 946.

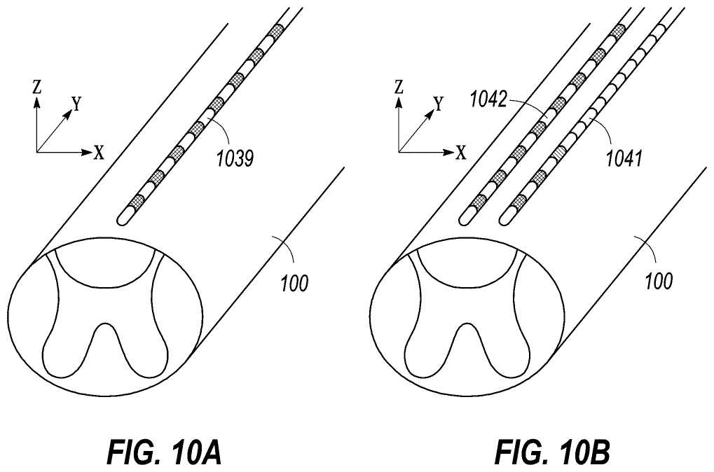

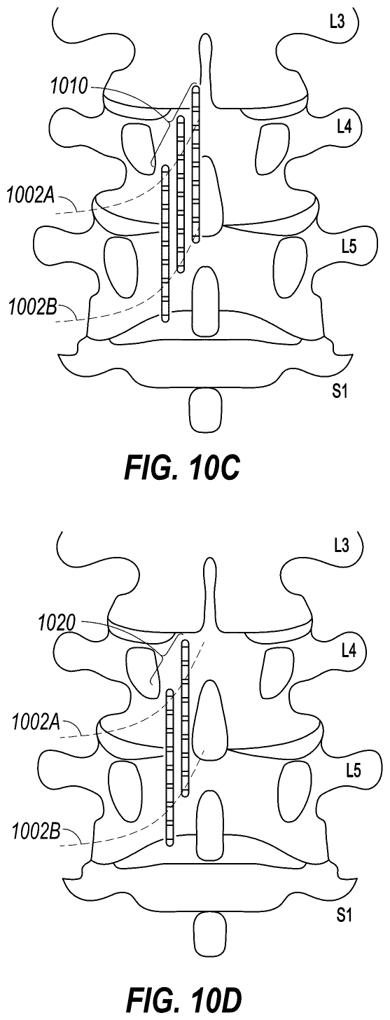

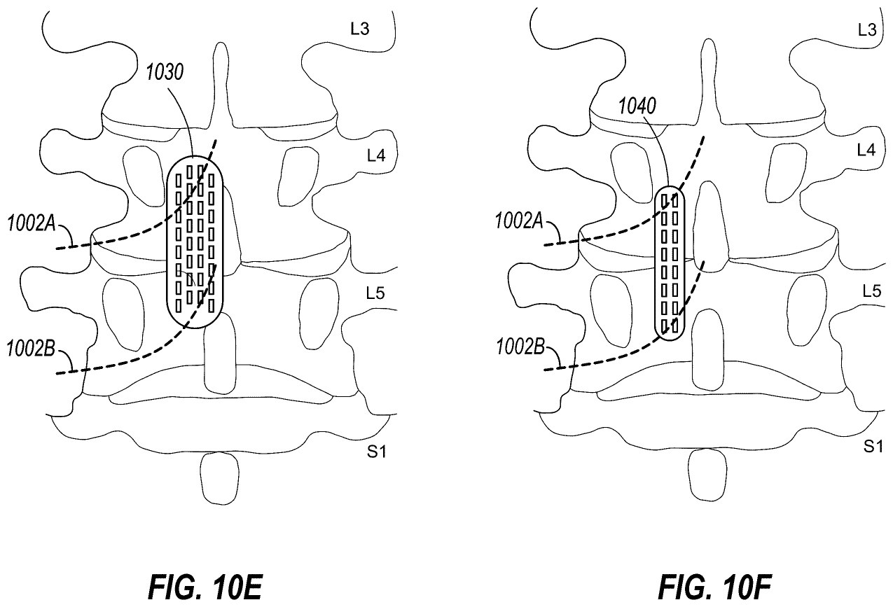

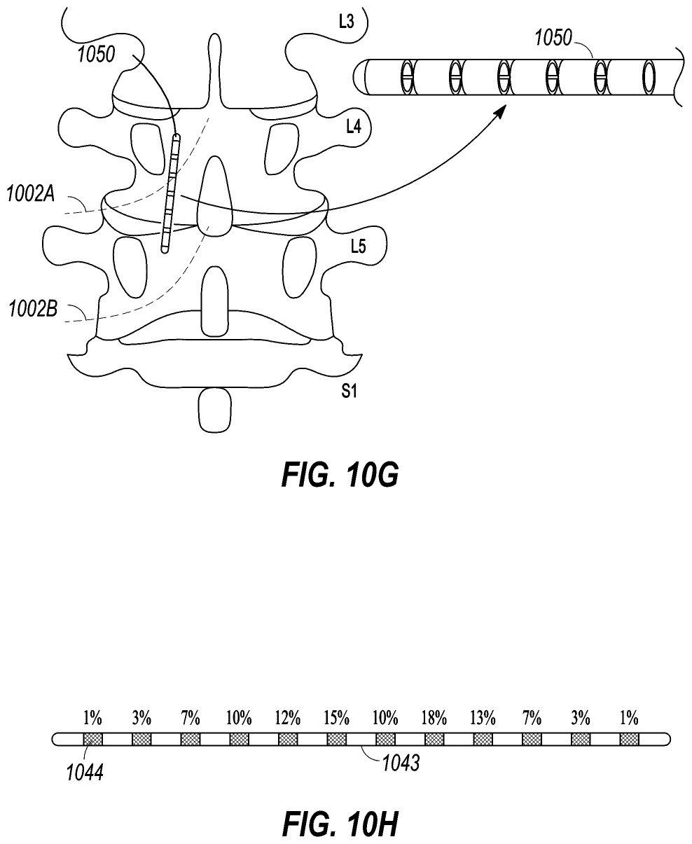

FIGS. 10A-10G are schematic views of embodiments of neuromodulation lead placement on a patient's spinal cord. Specifically, FIG. 10A is a schematic view of a single electrical neuromodulation lead 1039 implanted over approximately the longitudinal midline of the spinal cord 100. It is understood that additional leads or lead paddle(s) may be used, such as may be used to provide a wider electrode arrangement and/or to provide the electrodes closer to dorsal horn elements, and that these electrode arrays also may implement fractionalized current. FIG. 10B illustrates an embodiment where two electrical neuromodulation leads are implanted near the spinal cord. A first electrical neuromodulation lead 1041 is implanted more laterally with respect to the spinal cord, thereby placing it proximate the dorsal horn of the spinal cord. A second electrical neuromodulation lead 1042 is implanted more medially with respect to the spinal cord, thereby placing it proximate the dorsal column of the spinal cord 100.

Placement of the lead more proximal to the DH than the DC may be desirable to preferentially stimulate DH elements over DC neural elements for a sub-perception therapy. Lead placement may also enable preferential neuromodulation of dorsal roots over other neural elements. Any other plurality of leads or a multiple column paddle lead can also be used. Longitudinal component of the electrical field is directed along the y-axis depicted in each of FIGS. 10A-10B, and a transverse component of the electrical field is directed along the x-axis depicted in each of FIGS. 10A-10B. Some embodiments may include directional leads with one or more directional electrodes. A directional electrode may extend less than 360 degrees about the circumference of a lead body. For example, a row of two or more directional electrodes (e.g. “segmented electrodes”) may be positioned along the circumference of the lead body. Activating select ones of the segmented electrodes may help extend and shape the field in a preferred direction.

It is to be understood that additional neuromodulation leads or paddle(s) of the same or different types may be used, such as may be used to provide a wider electrode arrangement and/or to provide the electrodes closer to dorsal horn elements. In some examples, the neuromodulation leads or paddles maybe placed at regions more caudal to the end of the spinal cord, and the electrode arrays on the neuromodulation lead also may implement fractionalized current. FIGS. 10C-10G are schematic views of embodiments of neuromodulation lead placement on caudal regions of spinal column, such as the level of L3-L5 and S1, where virtually no spinal cord, but only dorsal roots, among other neural structures, are present. The neuromodulation leads or paddles may be placed medial or lateral to the spinal column, and proximal to one or more dorsal roots, and are configured to deliver modulation energy to the dorsal root fibers. In FIG. 10C, three percutaneous leads 1010 are positioned toward the left side of the spinal canal, and in FIG. 10D two percutaneous leads 1020 are positioned toward the left side of the spinal canal. In FIG. 10E, a single four-column paddle lead 1030 is positioned toward the left side of the spinal canal, and in FIG. 10F, a single two-column paddle lead 1040 is positioned toward the left side of the spinal canal. In FIG. 10G, a single percutaneous lead 1050 is positioned toward the left side of the spinal canal. In the illustrated example, the percutaneous lead 1050 includes multiple segmented electrodes that enable lateral control of the stimulation location via a single lead. Moreover, because the segmented electrodes are placed in close lateral proximity, they can be used to provide a high degree of lateral stimulation resolution.

While the examples illustrated in FIGS. 10C-10G show electrode lead placements to the left side of the spinal canal, these are by way of example and not limitation. In any of FIGS. 10C-10G, lead placements to the right side of the spinal canal may also be utilized. As can be seen from the figures, different types of leads with different numbers of electrodes and different electrode spacing (including different types than those shown) may be employed to provide dorsal root stimulation. These example lead placements differ from the placement of leads more proximal to the anatomical midline in traditional spinal cord stimulation (SCS) therapy.

The dorsal root trajectories 1002A and 1002B in FIGS. 10C-10G show that dorsal root fibers have different trajectories from dorsal column fibers, and they are not aligned with the anatomical midline. Accordingly, relative locations (e.g., lead entry angles) between the lead and the neural targets (e.g., dorsal column fibers or dorsal root fibers) can vary at different anatomical regions, as shown in FIGS. 10C-10G. When neuromodulation is specifically being targeted to dorsal root fibers, it is desirable to know the locations of the dorsal roots such that stimulation can be customized. The present document describes various embodiments of incorporating anatomy information of target neural tissue and known treatments for autonomic nervous system disorders into the process of stimulation field design and lead placement, which may help improve the neuromodulation precision and thus offer better a therapeutic outcome for such disorders.

FIG. 10H is a schematic view of an electrical neuromodulation lead 1043 showing an example of the fractionalization of the anodic current delivered to the electrodes on the electrical neuromodulation lead. These figures illustrate fractionalization using monopolar neuromodulation where a case electrode of the IPG is the only cathode, and carries 100% of the cathodic current. The fractionalization of the anodic current shown in FIG. 10E does not deliver an equal amount of current to each electrode 1044, because this embodiment takes into account electrode/tissue coupling differences, which are the differences in how the tissue underlying each electrode reacts to electrical neuromodulation. Also, the ends of the portion of the electrical neuromodulation lead include electrodes having lower gradient in the longitudinal direction. The magnitude of the electrical field tapers down at the ends of the electrical neuromodulation lead. Fractionalization of the current may accommodate variation in the tissue underlying those electrodes. The fractionalization across the electrical neuromodulation lead can vary in any manner as long as the total of fractionalized currents equals 100%.

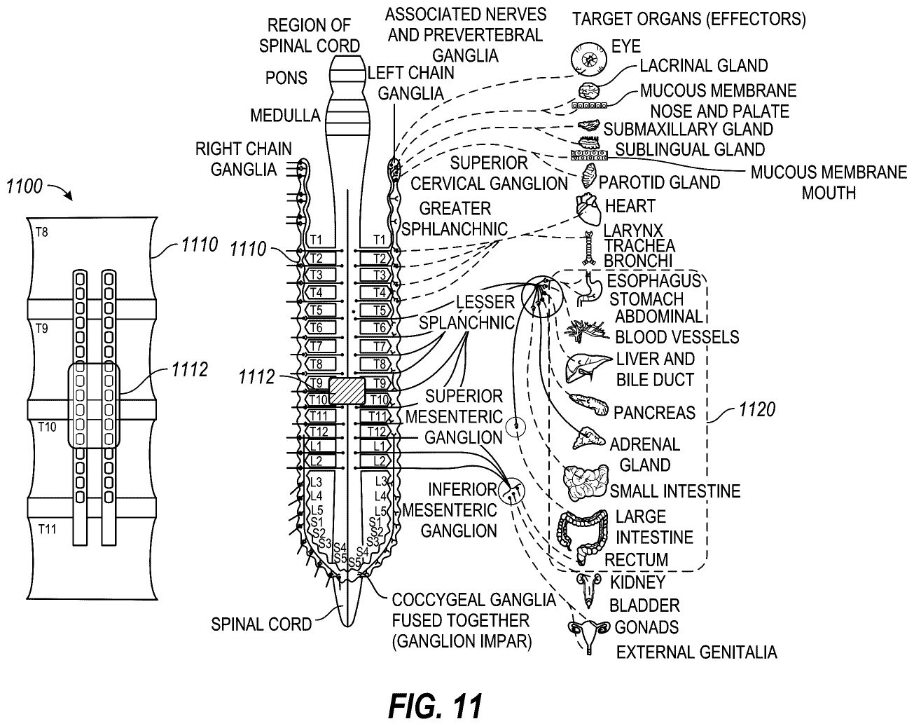

FIG. 11 is a diagram illustrating portions of the autonomic nervous system including spinal column origins (spinal levels) 1110 that provide spinal nerves to innervate various organs and tissues throughout the body, including, for example, blood vessels, stomach, intestine, liver, kidneys, bladder, genitals, lungs, pupils, heart, and sweat, salivary, and digestive glands. The innervating spinal nerves originate primarily from motor neurons in autonomic ganglia that are located in two paravertebral chains on either side of and parallel to the spinal cord. In patients receiving SCS, electrodes are placed at neural targets at certain spinal cord levels or spinal nerves (e.g., dorsal roots or dorsal root ganglion), and the stimulation of such neural targets may be used to mitigate or affect disorders or side effects specific to those organs or tissues innervated by the spinal nerves.

As illustrated in the exploded view 1100 of FIG. 11, in a patient implanted with a SCS lead system, stimulation energy (e.g., pulse train) delivered at selected electrodes such as electrodes 1112 at spinal levels T9-T10 may be used to affect the organs and organ systems 1120 innervated by the autonomous nerves originated from those spinal levels, including stomach, liver, adrenal glands, and small and large intestines. Such correspondence between the anatomical systems, organs, and tissues and the spinal levels (i.e., spinal column origins of the nerves innervating the respective organs and tissues) can be utilized to titrate a SCS configuration or dosing to alleviate autonomic symptoms, and to treat or alleviate autonomic disorders. Additionally, the correspondence between the anatomical systems, organs, and tissues and the spinal levels can be used for more effective surgical planning, simulation, and treatment of multiple disorders and anatomical systems.

In some of the emerging indications of SCS treatment, pain may not be the only problem or the most significant problem of patients who live with chronic or life-threating conditions. Clinicians who recommend and use SCS treatments, however, may not be aware of, or know how to, treat concomitant dysfunctions (e.g., secondary symptoms or effects in one or multiple body systems connected to the autonomic nervous system). SCS offers a variety of capabilities to improve the overall quality of life to patients affected by chronic conditions, including symptom reduction or therapies from stimulation effects delivered via peripheral nervous system stimulation.

The following proposes various method and system implementations to enable a robust evaluation of patients with multi-factorial disorders. These implementations include features that enables clinicians to guide SCS surgery planning and treatment, based on multiple types of symptoms and effects that SCS may be able to treat via the autonomic nervous system. The evaluation of multiple types of symptoms, effects, or other aspects of multiple body systems or conditions is referred to herein as a “multi-dimensional” or “multi-factorial” evaluation. The following also introduces approaches for follow-up treatments or adjustments on patients, by evaluating multiple dimensions of treatment that can provide a more holistic assessment of outcomes. As a result, many different types of SCS leads and electrodes can be placed and operated to treat multiple dysfunctions of a particular patient.

A variety of data may be considered in attempting to determine what is a “best” approach to treat a multi-factorial condition involving the autonomic nervous system. For instance, consider a chronic disease such as diabetes. Complications from diabetes in the cerebrovascular system may include brain stroke or cerebrovascular disease; complications in eye health may include diabetic retinopathy, cataracts, or glaucoma; complications in dental health may include periodontal disease (gum disease); complications in the cardiovascular system may include cardiovascular disease; complications in the vascular system may include peripheral vascular disease and foot damage; complications in the sensory system may include diabetic neuropathy; and complications in the renal system may include diabetic nephropathy. For instance, consider that in emerging indications of chronic health conditions, multiple problems are present besides pain. Likewise, consider a specific condition such as Diabetic Peripheral Neuropathy (DPN) or Chemotherapy-induced peripheral neuropathy (CIPN), which involves multiple autonomic complications and symptoms in the sensory system, motor system, and organ systems. The following enables planning and treatment for aspects of multiple characteristic medical conditions, including to identify uses of multi-port and multi-electrode SCS leads to more effectively treat conditions and symptoms associated with the autonomic nervous system.

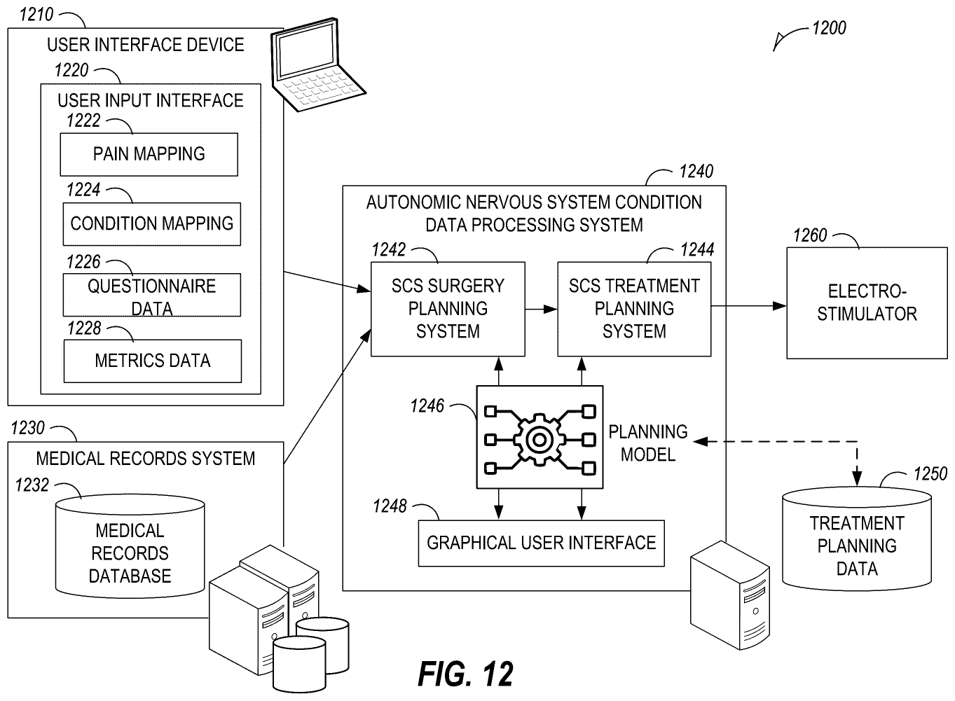

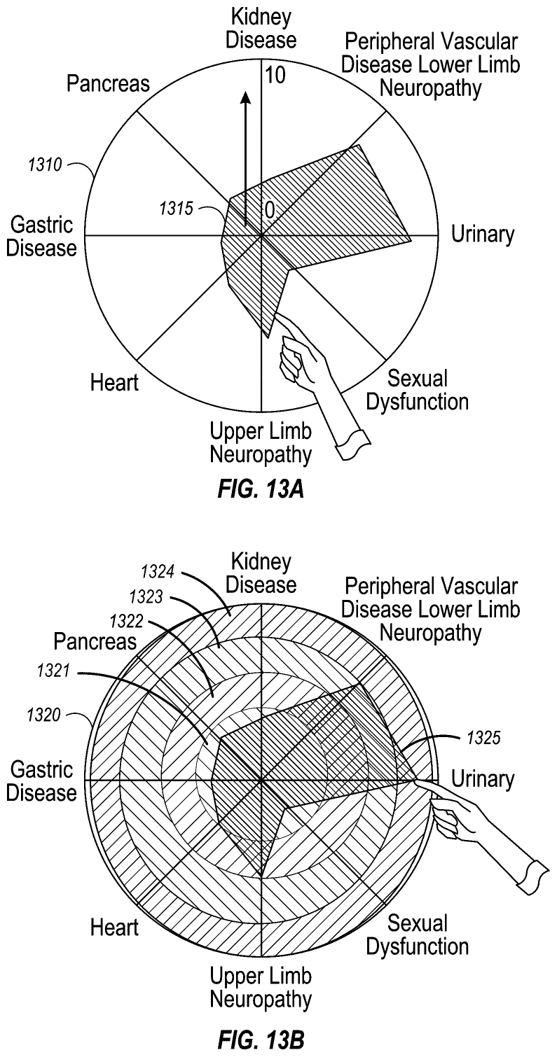

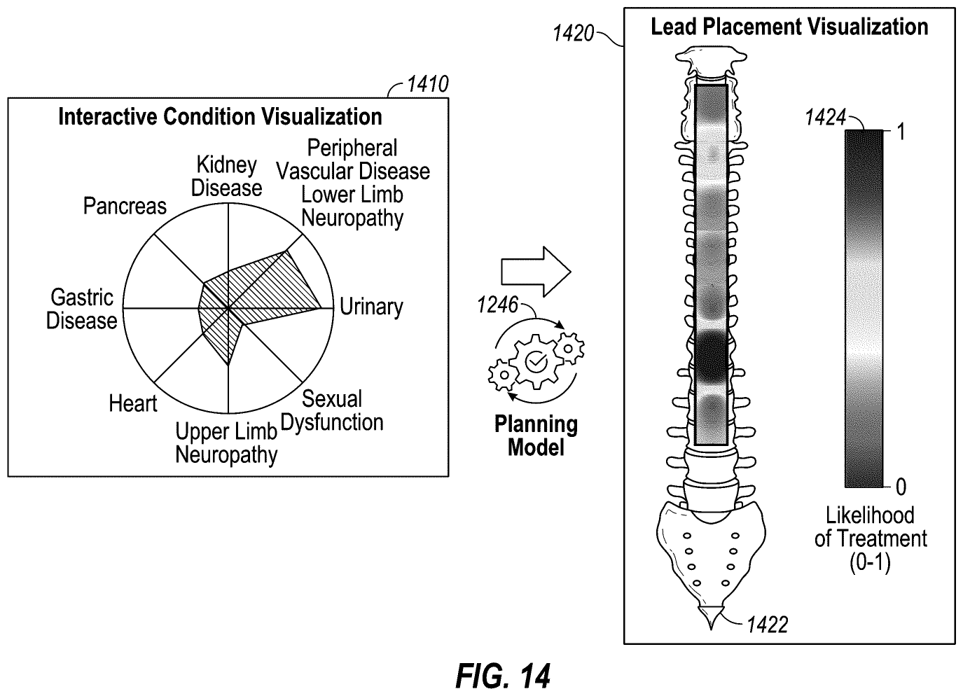

The techniques discussed below include determining how possible locations of peripheral nervous system stimulation are considered during surgical planning of a SCS system, to support prospective treatment for multiple aspects of autonomic nervous system conditions. The following also discusses techniques for assessing treatments and changes for multiple aspects of autonomic nervous system conditions after a SCS system is implanted. Such assessment is provided in some examples with the use of a SCS surgery planning system and a SCS treatment planning system, discussed with reference to FIG. 12. Such assessment is also provided in some examples with the use of a multi-dimensional input visualization and selection user interface, discussed with reference to FIGS. 13A, 13B, and 14, below.

FIG. 12 is a block diagram illustrating, by way of example and not limitation, a neuromodulation system 1200 configured to identify or provide neurostimulation to a neural target to reduce or alleviate autonomic symptoms associated with one or more medical conditions. In an example, the neuromodulation system 1200 can provide spinal cord stimulation (SCS) at a spinal neural target with use of an electrostimulator 1260, and can be designed for providing a pain management or medical condition management as its primary or secondary objectives. The system 1200 can automatically, or with user intervention, be used to deploy a stimulation setting on the electrostimulator 1260 to avoid or alleviate autonomic symptoms or conditions, separately or in combination with pain control in the patient.

The neuromodulation system 1200, which is an embodiment of the neuromodulation system 200, may include or be operably coupled to a user interface device 1210, a medical records system 1230, a data processing system 1240, a treatment planning data 1250 source, and the electrostimulator 1260. Portions of the neuromodulation system 1200 may be implemented in the implantable system 521 or the external system 523. In an example, the user interface device 1210 and the data processing system 1240 may be included in or operably coupled to a programming device, such as the programming device 410 or the CP 629.

The user interface device 1210 provides a user input interface 1220 for a user (e.g., patient, clinician, caregiver, or other interested party) to provide data relevant to one or more patient medical conditions. In an example, the user interface device 1210 can be a smartphone or personal computer executing a specialized software application to receive inputs as discussed below. In another example, the user interface device 1210 can be a client computing device to access and load a user input interface provided by a remote computing system such as a cloud-hosted website. In yet another example, the user interface device 1210 can be a programmer device, such as the CP 629, that allows a physician to receive inputs, in addition to other functions such as to consult with the patient to obtain information including pain relief and SCS-related side effects or symptoms, remotely review stimulation settings and treatment history, perform remote programming of the electrostimulator 1260, or provide other treatment options to the patient. In other examples, the user interface device can be an example of the programming device 220 or the programming device 410 as illustrated respectively in FIGS. 2 and 4, configured to allow a user (e.g., the patient, clinician, or a device expert) to interact with the neuromodulation system 1200 by providing input data values relevant to medical conditions or states, and other inputs for surgery planning and treatment.

The user interface device 1210 can collect data specific to autonomic symptoms or side effects (including pain) caused by medical conditions, and information that can be used to assess the severity or degree of such medical conditions. As illustrated in FIG. 12, the user input interface 1220 (e.g., a graphical user interface with input and output functionality) may provide user interface (UI) control elements to input and output information based on: pain mapping 1222 UI control elements and functionality; condition mapping 1224 UI control elements and functionality; questionnaire data 1226 UI control elements and functionality; and metrics data 1228 UI control elements and functionality. For example, the pain mapping 1222 may be used to identify pain experienced at different body areas associated with neural targets; the condition mapping 1224 may be used to identify symptoms or disfunctions of autonomic medical conditions that are experienced at different body areas associated with neural targets; the questionnaire data 1226 may be used to receive or review questionnaire data values related to pain or autonomic medical condition symptoms; the metrics data 1228 may be used to input or review measurements of various medical conditions or physiologies observed by sensors or by a human observer.

The user interface device 1210 can include a display (not shown) and other output device to present (e.g., textually, graphically, audibly) information such as the autonomic symptoms, the identification of the affected anatomy, and other pain and condition information. In an example, the user input interface 1220 may provide a simulation environment to identify, based on a list and measurement of symptoms, the systems, organs, or tissues likely to be affected or treated (e.g., when treated by SCS treatment). The user can select applicable symptoms and/or the affected anatomy from the list, such as via a UI control element on the user input interface 1220. In other examples, the user input interface 1220 may provide a graphical visualization and interaction tool to multiple dimensions of treatment (e.g., effects on different body systems) to be shown to and changed by a user. Examples of the user input interface 1220 relative to treatment and effects on different body systems, are discussed below with reference to FIGS. 13A and 13B.

The affected anatomy may include certain systems (e.g., bodily, head, cardiovascular, gastrointestinal, or urological systems) or organs (e.g., heart, stomach, intestines, bladder, endometrium, skin) where symptoms or side effects arise or persist from an autonomic condition. In some examples, the user may additionally provide information about the severity of a symptom or a side effect of the autonomic condition. The severity can take a numerical value (e.g., on a 1 to 5 scale) or a categorical value (e.g., “Mild”, “Moderate”, or “Severe”). The user may provide information on the autonomic symptoms and the affected anatomy in different formats, such as text, graphical, or verbal descriptions or annotations, among others. Interactive interfaces such as a voice agent or chatbot may also be used to collect information and control the collection of input. Examples of visualizations and data collection features of the user input interface 1220, including treatment and effects on multiple body systems or conditions, are discussed in more detail below with reference to FIGS. 13A and 13B.

In some examples, the user may additionally provide pain data or feedback on pain or symptom relief by SCS therapy via the user input interface 1220. The pain data or the feedback on pain relief in the pain mapping 1222 may include identification of pain sites, distribution of the pain, intensity of pain at various pain sites, or temporal pattern such as persistence of the pain at various pain sites, a pain drawing with pain markings identifying the locations, intensities, patterns of pain, among other information. In some examples, the user may provide information via the user input interface 1220 about patient health or medical information, such as change in medication, physical activities, medical procedures received, and the like. Such information, along with the condition mapping 1224, the questionnaire data 1226, and other metrics data 1228, may be used by the system 1200 to optimize neurostimulation lead placement and neurostimulation therapy delivered with the leads.

The system 1200 also shows the use of a medical records system 1230, which includes a medical records database 1232 that stores relevant medical data for a patient. The information from the user interface device 1210, the user input interface 1220, and the medical records system 1230 is provided to a data processing system 1240 for surgery planning and/or treatment planning, as discussed in the following paragraphs. The various options or prompts provided within the system 1200, or the types of models or algorithms used within the system 1200, may be determined from diagnoses or states indicated in the medical records database 1232.

The data processing system 1240 includes a SCS surgery planning system 1242 used to model and identify surgical planning for placement of neurostimulation leads in a patient, and a SCS treatment planning system 1244 used to model and identify neurostimulation treatment parameters for the patient with leads at a particular placement. The SCS surgery planning system 1242 and the SCS treatment planning system 1244 perform operations based on the use of a planning model 1246. The planning model 1246 may be provided by one or more artificial intelligence model, algorithm, rule set, or software system used to process inputs and provide outputs. The data processing system 1240 further includes a graphical user interface 1248 to interact with the SCS surgery planning system 1242 and SCS treatment planning system 1244, and optionally to affect the operation of the planning model 1246.

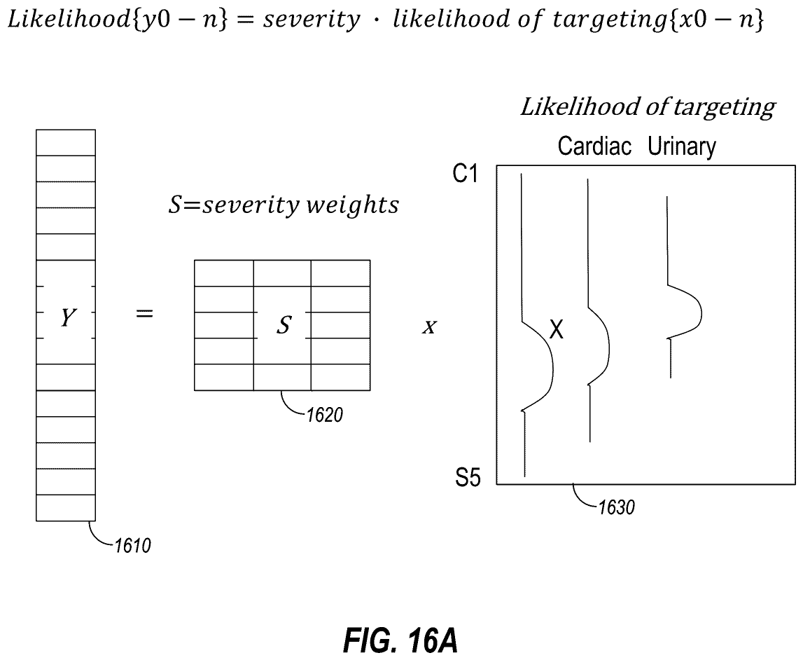

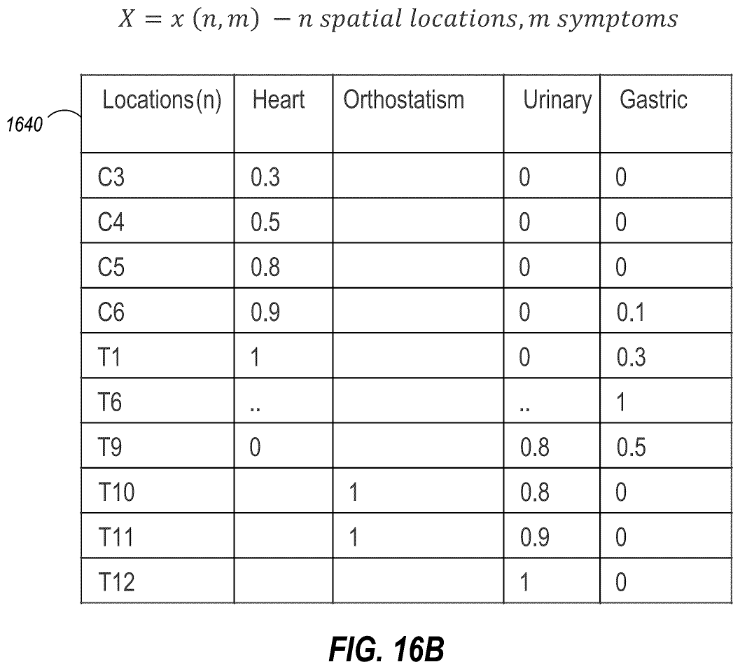

The planning model 1246 may further integrate with treatment planning data 1250. The treatment planning data 1250 in some examples may include historical or previously observed data values for treatments of particular autonomic nervous system conditions. The treatment planning data 1250 in some examples may include look-up tables, rules, data sets, or databases that associate medical conditions or treatment effects with particular neural targets, as discussed below with reference to FIG. 16B. The treatment planning data 1250 in some examples may also include weights, scores, biomarkers, or likelihood values for particular medical conditions and SCS treatments, as discussed below with reference to FIGS. 16A and 16B. In other examples, the treatment planning data 1250 may provide a source of training data or re-training data for the planning model 1246.

The SCS surgery planning system 1242 may be used to predict, simulate, or estimate treatment outcomes for autonomic nervous system conditions, dysfunctions, or symptoms based on the placements of respective leads for neurostimulation. Additional inputs and graphical simulations used with the SCS surgery planning system 1242, such as for visualizing the relationship between lead placement and condition treatments, are discussed with reference to FIG. 14, below.

The SCS treatment planning system 1244 may be used to determine stimulation settings for delivery with the simulated or actual lead placements. As used herein, a stimulation setting can be defined by a set of stimulation parameters with respective programmable or preset values. Examples of the stimulation parameters can include an electrode configuration (e.g., stimulation lead and electrode location, selection of active electrodes, designation of anode and cathode, and stimulation current or energy fractionalization across the electrodes), stimulation dose parameters (e.g., pulse width, frequency, pulse amplitude), stimulation pulse waveform, or an ON-OFF cycling of stimulation bursts (comprising a pulse train during an ON period, followed by a pulse-free period during an OFF period), among others. Such parameters may be generated directly or indirectly based on the user interaction with the visualizations discussed with reference to FIG. 13A and 13B, below. Such parameters may also be adjusted, modified, or weighted based on the approaches discussed with reference to FIGS. 16A and 16B, below.

The planning model 1246 may use the treatment planning data 1250 to more precisely identify neural targets to be modulated for symptom control, pain relief, or other therapeutic effects. Information retrieved from the treatment planning data 1250 can include a correspondence between the spinal column origins (spinal levels) and anatomical systems or organs innervated by the spinal nerves, information about the implanted leads including lead type (e.g., percutaneous leads, linear paddles, multiple-column paddles, or directional leads, among others) and lead placement and electrode locations, patient health status and medical history (e.g., change in medication, physical activities, medical procedures received), among other information. In a specific example, the treatment planning data 1250 may include a lookup table representing the correspondence between the spinal levels or lateral spinal targets and the corresponding anatomical systems or organs innervated by the spinal nerves originated from the respective spinal levels. An example of such a lookup table is discussed below with reference to FIG. 16B.

In further examples, the graphical user interface 1248 may include tools (e.g., UI control elements) that allow a user (e.g., the clinician) to predict, simulate, test, program, or modify the stimulation setting or placement for the SCS therapy or therapies, to improve pain relief effect and/or improve treatment of particular autonomic nervous system conditions. In some examples, the data processing system 1240 may use the planning model 1246 to automatically determine a “recommended”, “optimal” or “improved” stimulation placement, setting, or parameter value, based on medical condition states, and the identified affected anatomy received from the user. For instance, the graphical user interface 1248 may present to the user an “optimal” or improved stimulation setting or the recommended parameter adjustment based on multiple conditions and factors associated with the medical condition. The “optimal” or the improved stimulation setting or the recommended parameter adjustment may be based on interaction with the user interfaces discussed below with reference to FIGS. 13A and 13B.

The user can accept, reject, or modify the stimulation setting via the user input interface 1220. A stimulation setting is “optimal” or improved in the sense that neurostimulation delivered in accordance therewith is likely to achieve or improve a desired therapeutic outcome related to the medical condition, pain relief, or some secondary effect. In various examples, the “optimal” or recommended stimulation setting may include electrode configurations (e.g., monopolar, bipolar, or tripolar stimulation, anode and cathode designation); a location of central point of stimulation that represents a focal point of a stimulation field; one or more stimulation parameters (e.g., a current amplitude or a voltage amplitude, a pulse width, a pulse waveform, a pulse rate, a duty cycle); a modulation waveform continuously adjusting the amplitude, or the pulse width, or the frequency of the trains of pulses, where the modulation waveform can be a random signal, sinewave, triangular, exponential, logarithmic, quadratic, or any other modulating function. In an example, the stimulation setting may include ON-OFF cycling of stimulation bursts that comprises a pulse train in an ON period, followed by a pulse-free period in an OFF cycle.

In some examples, the stimulation setting may include paresthesia-based stimulation, which may cause paresthesia sensation during stimulation. Examples of the paresthesia-based stimulation may include a monopolar stimulation mode, a bipolar stimulation mode, a tripolar stimulation mode, a steering mode, a Sensations mode, and a rotation mode. The stimulation may be cathodic or anodic. In an example, monopolar anodic stimulation may be applied after a search using Multiple Independent Current Control (MICC) through the different electrode-tissue contacts to refine the size and shape of a stimulation field, and to customize therapy for individual patients. In an example, monopolar anodic stimulation may be applied with Time-Variant Pulses (TVPs), such as defined as rate, pulse width, or amplitude modulated with a specific function, such as a sinusoidal wave function, a random function following a statistical distribution (e.g., a Poisson distribution, or a uniform distribution), or other arbitrary waveforms. TVPs with monopolar anodic stimulation may be applied after a “sweet spot” search is done using MICC through the different contacts. A sweet spot is a desirable or optimal location for the neuromodulation field. In an example, a test region may be primed with the sub-perception neuromodulation field, and a sweet spot can be identified as a neural tissue that is therapeutically effective when targeted with sub-perception neuromodulation. The sweet spot test may involve a manual process to reprogram the neuromodulation field parameter set with different values to change the targeted location of the neuromodulation field. In some embodiments of the test, the targeted location may be automatically changed (e.g. trolled) by automatically changing values of the neuromodulation field parameter set. Some embodiments may semi-automatically change values of the neuromodulation field parameter set to change the targeted location of the neuromodulation field. In an example, monopolar cathodic stimulation may be applied with or without the TVPs. In another example, bipolar stimulation may be applied with or without TVPs. The bipolar configuration comprises an anode located at the rootlets, and a cathode located in the mid-lead. In an example, a tripolar stimulation may be applied in the rostrocaudal direction. In some examples, shunting cathodes may be used in MICC fashion to make anodic stimulation more localized (e.g., along rostro-caudal and medo-lateral direction). Because the rootlets span out at this point, the cathodes can be used to shunt away the anodic current from the rootlets that do not correspond to the rootlets of interest. In an example, a long rostro-caudal anodic monopole may be used to excite a larger region of the DREZ.

In an example, the stimulation setting may include paresthesia-free stimulation, which generally may not cause paresthesia sensation during stimulation. Examples of the paresthesia-free stimulation may include a Fast-Acting Sub-perception Therapy (FAST) mode, a Dorsal Horn Modulation (DHM) mode, a burst mode, and a Low-Rate Active Recharge (LRAR) mode. The FAST mode allows stimulation pulses to be delivered to provide profound paresthesia-free pain relief in a short time period (e.g., several minutes) by increasing surround inhibition. The DHM a stimulation mode that can target inhibitory interneurons over dorsal column fibers. Under the LRAR mode, sub-perception stimulation pulses are delivered at lower frequencies than the typical DHM frequencies.

Consideration of multi-dimensional aspects of complex medical conditions may be used to identify or change treatments of such conditions in multiple body systems or organs. As one example, a circular graph in the shape of a “wheel” with multiple “spokes” on each data axis may be provided as a visualization tool for balancing the severity or impact on respective characteristics of an autonomic medical condition. A circular graph provides an easy-to-understand approach to assess, quantify, and visualize multiple dimensions of patient health and conditions, for characteristics such as symptoms, problems, or side effects occurring in different body systems or organs. As explained below, FIGS. 13A and 13B provide specific examples of an interactive, circular graph, to identify the importance or severity of respective characteristics to a particular autonomic condition or treatment. These examples of a circular wheel are provided for illustration purposes, and it will be understood that other types of graphs or charts to present data in multiple dimensions or axes can be used with the present approaches.

FIG. 13A depicts a representation of a circle graph 1310 in the shape of a wheel, where values charted at different dimensions of the graph correspond to a severity value that is ranked within some range of values. Here, the range of possible severity values is depicted between values 0-10, for each domain (one of eight domains charted onto the circle graph 1310). Additional or fewer domains may be depicted within a circular graph or similar graph shapes. The circle graph 1310 may also be referred to as a “radar” plot. Other similar examples of circular graph shapes include a rose diagram, a polar area chart, a circular column, and the like. Other shapes and types of multi-dimensional graphs and charts may be presented.

Dimensions can be arranged next to each other on the circular graph in a topographic order, or based on some criteria or relationship. Charted dimensions can correspond to multiple factors, symptoms, organs, or conditions (e.g., as shown, where one of the dimensions corresponds to related conditions “Peripheral Vascular Disease” and “Lower Limb Neuropathy”). In some examples, the dimensions presented on the circle graph 1310 are unique to the particular patient, based on input medical data or clinician selections. In other examples, the circle graph 1310 may depict dimensions that are disease-specific, to allow evaluation between different symptoms or effects of a specific disease on particular organs.

FIG. 13A further depicts a user interaction with a custom-shaped data area 1315 of the circle graph 1310, which uses the data area 1315 to measure and represent severity in each dimension (e.g., severity values in each of the eight depicted characteristics, “Kidney Disease”, “Peripheral Vascular Disease/Lower Limb Neuropathy”, “Urinary”, “Sexual Dysfunction”, “Upper Limb Neuropathy”, “Heart”, “Gastric Disease”, “Pancreas”). For instance, a user interaction with a graphical display of the circle graph 1310 on a touchscreen may be provided by stretching and contracting the shape of the data area 1315. The severity score for each dimension can itself be based on multiple factors, such as biomarkers, scales, etc. Thus, the severity score for each dimension may represent a composite score that is calculated based on symptoms, to help identify the most severe conditions to be treated with SCS.

The visualization can also be used to display a patient status, such as the effects or impact on different of the results of a recommended or modified SCS treatment. For instance, a visualization can be projected on the circle graph 1310 to show how stimulation effects can provide symptom relief or treatments on the lower limb neuropathy and urinary system. In this setting, the numerical scale 0-10 can be used to represent beneficial effects or reduction in symptoms.

FIG. 13B depicts a similar representation of a circle graph 1320 in the shape of a wheel, but which includes color, shading, or gradients to emphasize different levels or ranges of effects. For example, the circle graph 1320 may use ranges within a colormap to depict ranges of severity. As a simple example of colors, severity may be indicated in ranges 1321, 1322, 1323, 1324 that correspond to shades of green (0-2.5), yellow (2.5-5), orange (5-7.5), and red (7.5-10)). An individual dimension-specific data area 1325 can overlay the wheel with a different color, shading, or gradient (e.g., with a partially transparent blue color). Other levels of transparency or opacity may be used to assist viewing and interaction with the values on the circle graph 1320.

The dimensions, ranges, and values within either circle graph 1310, 1320 can be informed based on different sources of data, including the pain mapping 1222, condition mapping 1224, questionnaire data 1226, metrics data 1228, or medical records database 1232, discussed above. Accordingly, the initial inputs to the data represented in the circle graph 1310, 1320 may be based on clinician input (e.g., values derived from objective metrics, scaled scores, ratings, etc.), patient input (e.g., values derived from symptom questions), algorithm-detected values, model-recommended symptoms or conditions to treat, etc.

In addition to providing an output that represents calculated or generated data values (e.g., to depict possible effects of treatment), the circle graph 1310, 1320 can be used as an input source for customizing or changing a treatment. For instance, user interaction with the circle graph 1310, 1320 can be used to adjust SCS treatments that occur at particular areas, and to change or switch certain types of SCS treatments or treatment effects in particular body systems.

The circle graph 1310, 1320 also can be used to track or compare the patient outcome after a SCS treatment has been applied, in a variety of user interface settings. In a first example, a web-based user interface may be used by a clinician to display and receive input with the circle graph 1310, 1320. This web-based user interface may be used to plan treatment and identify expected outcomes for a patient-specific treatment. In a second example, clinical programmer or a patient application on a smartphone or personal computing device may also present the circle graph 1310, 1320 to monitor recommended values associated with an ongoing or modified treatment. The presently described systems and methods also may be used to monitor the patient use of a neurostimulator and coordinate the collection of patient data. Patient data may be collected and analyzed to provide additional (updated) information for status plots and algorithms (e.g., subsequently presented to the clinician or the patient). Finally, the presently described systems and methods may be used to identify, change, and present patient-specific SCS recommendations to both patients and clinicians based on historical autonomic symptoms analysis.

FIG. 14 depicts a scenario where data values in a patient-specific, interactive condition visualization 1410 (e.g., a circular graph similar to 1310, 1320, discussed above) are converted to a lead placement or treatment visualization 1420. In this example, a multi-dimensional visualization is provided using a heat map or color scale map to depict the effect of SCS treatment or and treatment recommendations, such as for surgical lead placement planning or for creating or adjusting a stimulation field. Based on the severity values represented in the interactive condition visualization 1410, the lead placement or treatment visualization 1420 provides a spatial heat map. The values of the heat map include colors, gradients, or shading that correspond to a likelihood of treatment 1424 of the multiple dimensions with neural targets at locations of a spinal cord 1422.

In this example, the algorithm of the planning model 1246 can weigh multiple factors or criteria from the interactive condition visualization 1410, such as symptoms to be treated, severity of a condition, and neural target locations within the spinal cord. Each symptom dimension can have an associated region of SCS treatment likelihood (e.g., the likelihood that an SCS treatment at a particular location will affect a particular symptom). The data values for SCS treatment likelihood at a particular neural target for a particular symptom may be based on a spatial data table or matrix, as discussed in more detail below.

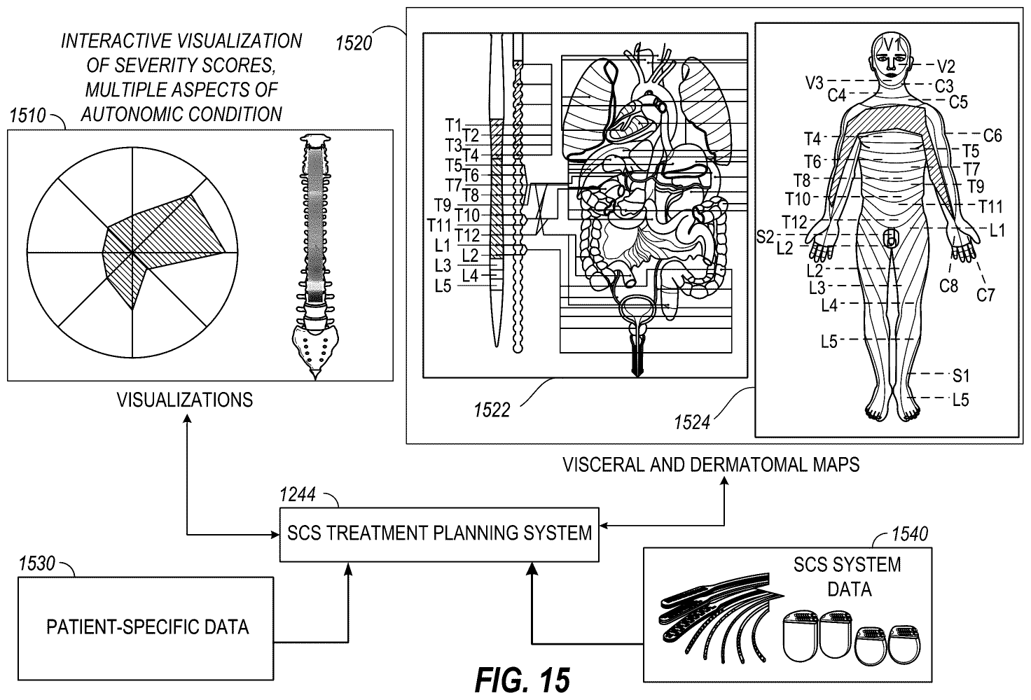

FIG. 15 provides an overview of other types of data inputs and outputs that may be used with the SCS treatment planning system 1244. These data inputs and outputs may be provided through various data values and user interfaces, including the user input interface 1220 and graphical user interface 1248, or variations of the visualizations (e.g., 1310, 1320, 1410) discussed above. Here, particular treatments can be personalized to a particular patient with the SCS treatment planning system 1244 on the basis of multiple patient characteristics provided in the patient-specific data 1530, or interactions received (e.g., inputs by a clinician or a patient) with interactive visualizations such as a circle graph visualization 1510 or anatomical maps 1520.

The SCS treatment planning system 1244 may include a neurostimulation targeting simulator that presents one or more types of interactive anatomical maps 1520. In an example, the anatomical maps 1520 include a visceral map 1522 to provide a visual representation of viscerae (internal organs) connected to neural targets of the spinal cord, including a mapping of stimulated spinal cord locations to particular organs, organ groups, or anatomical body systems. The effects of the neurostimulation on the organs, organ groups, or anatomical body systems connected to the stimulated spinal cord locations may be shown with shading, colors, or other visual effects on respective organs. In an example, the anatomical maps 1520 also include a dermatomal map 1524 to represent the potential dermatomes (areas of skin in which sensory nerves derive from a spinal nerve root). Thus, in the visceral map 1522 and in the dermatomal map 1524, designated areas (and colors, shading, gradients, or other emphasis) can indicate the simulated results of neurostimulation targeting based on a planned or recommended lead configuration and placement of the lead on the spinal cord for the particular patient.

The SCS treatment planning system 1244 may also provide interactive abilities to simulate the activation of different types of leads and lead placements on patient anatomy, including to provide updated effects in the visualization 1510 such as on a circle graph or spinal cord heat map. The simulation of the lead placement may be customized using SCS system data 1540 and patient-specific data 1530, including information regarding what neurostimulator is implanted in the patient, existing lead and electrode locations, the state of various medical conditions, etc. In further examples, the visualizations 1510 or maps 1520 may be customized to a patient's particular anatomy, medical conditions, or types of treatments.

The SCS treatment planning system 1244 can operate one or more algorithms (e.g., implemented with the planning model 1246) to determine areas or effects to highlight or emphasize (e.g., with color, shading, labels, annotations, etc.) in the visualizations 1510 and maps 1520, to identify the particular anatomical areas or organs being treated for a particular autonomic nervous system condition. As discussed above, the spatial algorithm or other aspects of the planning model 1246 may perform analysis based on a number of programmed, modeled, trained, or analyzed data values. The highlight or emphasis can be based on a likelihood of treatment or beneficial effects, based on physiology and current understanding of SCS targeting and treatments. Such graphical user interfaces can simulate the result of activating particular lead placements and provide useful feedback to demonstrate how SCS can have beneficial effects on the peripheral nervous system (and the associated autonomous nervous system conditions).

Neurostimulation lead placement, device settings, and programming can be recommended based on the gathered information, and as a result of the interactive simulation of effects. For instance, a clinician can use the treatment planning system 1244 to identify particular spatial targets (e.g., dorsal columns, roots, dorsal horn), frequencies (e.g., 40 Hz or 500 Hz), pulse width (e.g., 200 μs or 400 μs), and treatment paradigms (e.g., sub-perception or supra-perception) based on the simulation and interaction with the visualization 1510 and maps 1520. Other mechanisms for representing or modifying the stimulation settings or values (e.g., using the interactive anatomy representations) may also be provided.

FIGS. 16A and 16B provide simplified examples of data values and calculations used with the approaches above. FIG. 16A provides an example of data modeling used to determine spatial values for SCS treatment. FIG. 16B provides an example table of data values used at respective spatial locations with multiple characteristics (e.g., each characteristic corresponding to a respective dimension). A table of data values (e.g., as shown in FIG. 16B) is used for mapping neural targets based on a likelihood of targeting a particular characteristic of a medical condition (e.g., as shown in FIG. 16A). The table of data values may be populated with values based on literature or research, and may be updated to become more accurate based on population-based learning. Likewise, the table of data values may be individualized once a patient obtains treatment.

In FIG. 16A, a vector of data values Y 1610 represents the different spatial points (e.g., neural targets) in the spinal cord. Y can correspond to a variety of granular or dense data values, such as a simple categorization that corresponds to 33 vertebrae levels, or that corresponds to a more detailed categorization of hundreds of regions, or that corresponds to an even more complex categorization of a dense spatial map tracking thousands of individual pixels. Thus, the vector 1610 provides values for y0−yn=spatial coordinate in a spinal cord.

Each location in the vector 1610 y[y(n)], is “scored” for targeting, based on the application of severity weights S 1620 to a likelihood of targeting 1630. This weights the severity of each spatial location depending on patient-specific symptoms. The severity weight provides a composite severity score of the different ways that a function or organ can be measured, based on subjective information provided by a patient, objective test data, etc. This enables the values in y to represent which spatial locations/vertebral levels should be prioritized for neurostimulation.

Turning to FIG. 16B, the spatial matrix X 1640 contains values for the n spatial locations determined in y, to account for m organs, functions, or symptoms. Values in the spatial matrix X 1640 are intended for spatial information. For example, the “heart” column could be represented as a gaussian with center in T1-T3 and slowly decay around. This information will be weighted by the severity of “heart” issues for the particular patient. This way, if the patient has heart issues, his or her final scores around T1-T3 will be high, suggesting the use of electrostimulation at this location. Similar values may be calculated for other conditions involving pain, urinary, gastrointestinal, etc. For instance, if a patient has pain in low back and urinary problems, the scoring will produce high y scores at T9-10, and so forth. Although the table of data values shown with spatial matrix X 1640 is shown on a vertebra level, this table may also be spatially granulated (based on finer segmentations or pixels) corresponding to the information represented in the spatial map.

For each system/symptom, a composite severity score {e.g., 0-10} can be calculated based on clinical data (e.g., questions) and physiological data (e.g., biomarkers, etc.). As non-limiting examples, scores may be based on composite scores used for clinical evaluation or rating, such as: Orthostatic Intolerance Score; Cardiorespiratory Score; Gastrointestinal Score; Urinary Score; Genital/Sexual Function Score; Sudomotor Function Score; Pupillomotor Function Score; Vasomotor Function Score. The list of symptoms associated with a particular anatomical system or organ may be determined based on data collected (e.g., patient survey) from a patient population, or customized by a clinician. A custom questionnaire could be deployed based on symptoms at each function (e.g., cardiovascular, gastrointestinal, urinary, genital, bodily, head) or body region (e.g., pain in dermatomes).

The list of symptoms may provide questions or data inputs related to symptoms, including type of symptoms, severity, and frequency. For example, the gastrointestinal symptoms may include constipation, diarrhea, or gas and bloating. In another example of gastrointestinal symptoms, the symptoms may relate to visceral pain of one or more internal organs, such as bladder pain, endometriosis, irritable bowel syndrome, and prostate pain. The patient may select applicable symptoms from the displayed symptoms (e.g., constipation for example), such as by tapping on or activating a corresponding selection option of the symptom. In further examples, the user may provide additional characterization or description of the identified symptom, such as symptom severity. The severity can have a numerical value (e.g., on a 1 to 5 scale) or a categorical value (e.g., “Mild”, “Moderate”, or “Severe”). Thus, the evaluation of particular conditions or body systems can include simple ratings (e.g., symptoms corresponding to a rating of severe, moderate, mild, none).

In further examples, the SCS surgery planning system 1242 or the SCS treatment planning system 1244 can consider the state of multiple anatomical systems, as indicated by patient symptom information, when determining placement of an SCS lead or treatments using placed leads. For instance, symptoms and severity of each organ/system can be graded using standard questionnaire responses. As an example, the Boston Autonomic Questionnaire uses a composite severity score for each domain using patient-oriented questions. For example, consider the following scoring scale for the following physiology functions:

TABLE 1

Part 1: Orthostatic Intolerance Score
Add questions 1.-1.3 and divide by 3.
Part 2: Cardiorespiratory Score
Add questions 2.1-2.3 and divide by 3
Part 3: Gastrointestinal Score:
Add questions 3.1-3.7 and divide by 7
Part 4: Urinary Score:
Add questions 4.1-4.4 and divide by 4
Part 5. Genital/Sexual Function Score
Part 5: Males Add questions 5.1-5.3 and 5.5 and divide by 4
Part 5: Females Add questions 5.4 and 5.5 and divide by 2
Part 6: Sudomotor Function Score:
Add questions 6.1-6.3 and divide by 3
Part 7: Pupillomotor Function Score:
Add questions 7.1-7.2 and divide by 2
Part 8: Vasomotor Function Score:
Add questions 8.1-8.3 and divide by 3

As an example, questions for a particular body system (Urinary system) which produces a particular body system score (Urinary score) may include the following questions:

TABLE 2
4.1: Do you urinate frequently?
Scale -- 0: Never to 10: Always
4.2: Do you wake up at night in order to urinate?
Scale -- 0: Never to 10: Always
4.3: Do you urinate urgently to avoid losing control of your urine?
Scale -- 0: Never to 10: Always
4.4: Do you have difficulty starting your urine flow?
Scale -- 0: Never to 10: Always

Other non-limiting examples of autonomic conditions are discussed below. For example, chronic kidney disease (CKD) severity can be based on the blood biomarkers glomerular filtration rate and albumin. Based on these values (e.g., table cutoffs) the severity can be projected onto 0-10 scale as follows.

TABLE 3

CKD{0 − 10} = a1 * GFR + a2 * Albumin + . . . + e

Similar approaches can be used to assess the severity of other medical conditions. For Gastrointestinal conditions, the Leeds Dyspepsia Questionnaire (or the Short Form Leeds Dyspepsia Questionnaire) can be used to ask a number of questions related to the frequency and severity of dyspepsia. An electrogastrogram also may provide an objective assessment. For bowel conditions such as constipation, the Frank et al. 2001 self-reported constipation questionnaire or physical exam results may be considered. For irregular bowel movement or diarrhea, the Lui et al. 2014 Diarrhea questionnaire or physical exam results may be observed. Symptoms that occur in abdominopelvic quadrants may be observed and recorded.

For Orthostatism, an Orthostatic Hypotension Questionnaire (OHQ) may provide a measurement of orthostasis. The results of an objective test, such as a Tilt Table Test, may also be observed and recorded.

For urinary conditions, e.g., involving the Bladder, a Urinary Distress Inventory, Short Form (UDI-6) Overactive Bladder Questionnaire (OAB-q) or OAB-BAT Incontinence Impact Questionnaire short form IIQ-7, may provide a measurement of urinary disorders. For Erectile dysfunction, a disorder measurement may be provided by the International Index of Erectile Function (IIEF).

For pancreas function conditions, one or more of general pancreatic function, beta cell specific, or inflammation measurements may be used. Other measurements may include quantitating the degree of insulin resistance (e.g., the euglycemic hyper insulinemic clamp procedure). These measurements may also be coordinated with standard blood-glucose measurements.

For peripheral circulation conditions, a questionnaire such as NRS (a numerical rating scale) or PADQOL (peripheral artery disease-specific quality of life questionnaire, as adjusted) that relates to peripheral artery disease, can be used. Measurements from an objective assessment may be provided by one or more of: Ankle-brachial index; Toe-brachial index; Peripheral Arterial Duplex; Imaging Assessment; Transcutaneous oximetry (tcpO2); Thermography; or Pulse Volume Recording (PVR).

For Pain-Neuropathy conditions, an objective assessment may be provided by: Pittsburgh Sleep Quality Index; or a measurement on the Neuropathic Pain Scale, (NRS). A Functional assessment may be provided by: Oswestry Disability Index; Profile of Mood States questionnaire (POMS); or European Quality of Life Five-Dimensional Five-Level questionnaire.

It will be understood that aspects of the visualizations and data processing may be implemented in different types of computing systems and platforms. For example, in a clinical software implementation, the core data processing software may be provided by a Web/Desktop application that is connected to, and updated by, a mobile phone application (which provides an application that is an extension of the core software). In this setting, data values and visualizations may be computed in the cloud. In a patient software implementation, a patient may be provided with data input access, potentially via another patient software app. The core data processing software may also be linked to online patient questionnaires provided by a third party (e.g., hospital-mediated) platform. The core data processing software can also receive information directly from a patient implanted device (e.g. via a CP or IPG).

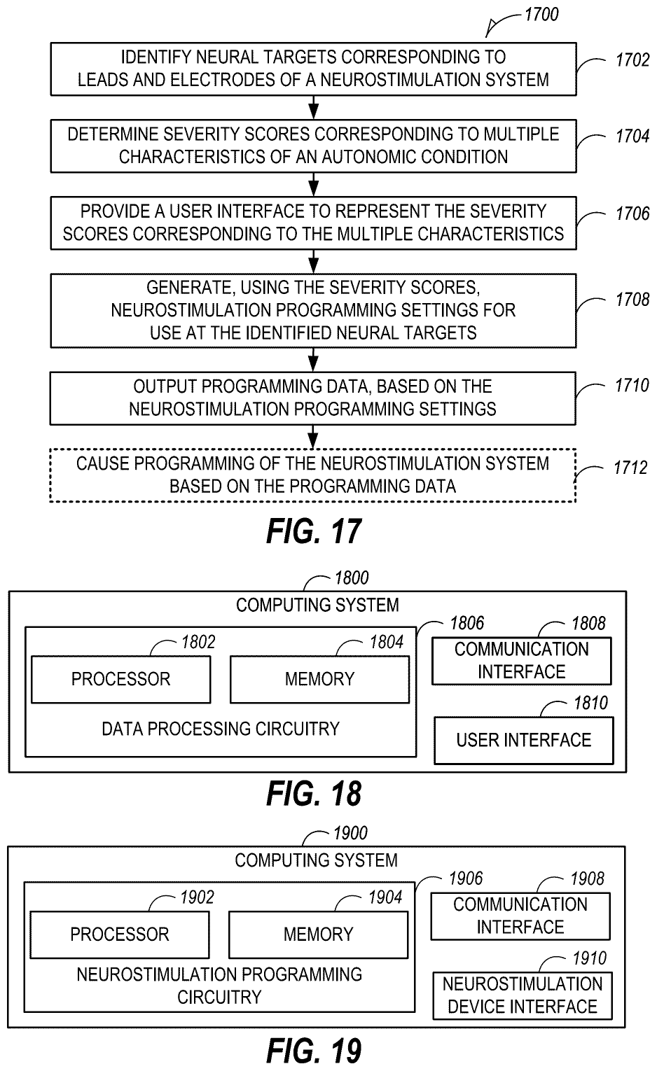

FIG. 17 illustrates, by way of example, an embodiment of a processing method 1700 implemented by a system or device for determining neurostimulation programming for a patient and patient autonomic condition. For example, the processing method 1700 can be embodied by electronic operations performed by one or more computing systems or devices (including those at a network-accessible remote service) that are specially programmed to implement data analysis and/or neurostimulation data processing operations. In specific examples, the operations of the method 1700 may be implemented through the systems and data flows depicted above, at a single entity or at multiple locations.

In an example, the method 1700 begins at 1702 by identifying neural targets corresponding to leads and electrodes of a neurostimulation system. This may include information on a neurostimulation system planned to be implanted or already implanted in a particular patient. In an example, the neural targets of the patient include spinal neural targets, and the neurostimulation includes spinal cord stimulation (SCS) to be provided via the respective leads and electrodes to the spinal neural targets.

The method 1700 continues at 1704 by determining respective severity scores corresponding to multiple characteristics of an autonomic condition. These severity scores may be determined based on input received from a user interface device that receives patient data related to the autonomic condition of the patient. For instance, the respective severity scores may be determined based on the patient data received in a symptom questionnaire, with the symptom questionnaire providing a numerical measurement of effects of the autonomic condition in respective anatomical systems or organs that correspond to the multiple characteristics of the autonomic condition. In further examples, additional patient data is identified from at least one medical record, and some of the multiple characteristics of the autonomic condition are identified based on the additional patient data.

The method 1700 continues at 1706 by providing (or, communicating with a client device that provides) a user interface (e.g., at a user interface device) to represent the severity scores corresponding to the multiple characteristics. In an example, this user interface comprises a graphical user interface to receive patient data (e.g., relating to the autonomic condition) and output severity data (e.g., relating to the severity scores). For example, the severity data may be communicated to the user interface device, which enables the user interface device to output a visualization of the respective severity scores (e.g., in a graphical user interface) that depicts relative severity for each of the multiple characteristics of the autonomic condition. In a further example, the respective severity scores are represented in a visualization (e.g., in a graphical user interface) within a defined range or groups of ranges for each of the multiple characteristics of the autonomic condition.

As noted above, the visualization of the respective severity scores may include a circular graph that positions the multiple characteristics of the autonomic condition at respective locations in the circular graph. This circular graph may include an overlay area that maps the respective severity scores onto the respective locations in the circular graph. This circular graph also may receive user interaction to change the respective severity scores at the respective locations in the circular graph. In other examples, the user interface device may output: an anatomical visualization including at least one visceral map or dermatomal map, which provides corresponds to symptoms or treatment effects at the neural targets; or, a heat map corresponding to symptoms or treatment effects at the neural targets, with this heat map being overlaid on the neural targets on a visualization of a spinal cord for the patient (or, other anatomical areas).

The method 1700 continues at 1708 by generating, using the severity scores, neurostimulation programming settings for use at the identified neural targets. In an example, neurostimulation programming settings are determined based on a mapping of the neural targets to a severity score value (e.g., corresponding to each of the multiple characteristics of the autonomic condition). In a further example, mapping of the neural targets is based on a score computed from a likelihood of targeting a respective characteristic of the autonomic condition and a severity weight for the respective characteristic of the autonomic condition.

The method 1700 continues at 1710 by outputting programming data, based on the neurostimulation programming settings. In specific examples, the neurostimulation programming settings includes information for: one or more spatial targets, one or more frequencies, and one or more pulse-widths used for the neurostimulation. Such neurostimulation programming settings may be generated based on a configuration of the respective leads and electrodes of the neurostimulation system used for the patient, based on a configuration that provides specifications for: a type of implantable pulse generator, a type of lead, a number of leads, and a number of electrodes on respective leads.

In further examples, the method 1700 continues at 1712 by causing programming of the neurostimulation system, based on the programming data. In specific examples, such programming is provided with generated programming instructions for an electrostimulator of the neurostimulation system, with the programming instructions corresponding to the neurostimulation programming settings to control stimulation of at respective neural targets of the patient via the respective leads and electrodes. In specific examples, the programming instructions includes one or more stimulation parameters including: an electrode configuration; one or more stimulation pulse parameters including a pulse amplitude, a pulse width, or a stimulation frequency; a stimulation pulse waveform; an ON-OFF cycling scheme comprising an ON period for delivering stimulation pulses and a subsequent stimulation-free OFF period; or a charge per second (CPS) or a charge per hour (CPH) delivered to the a respective neural target. Other approaches for programming or outputs may be provided consistent with the examples above.

FIG. 18 illustrates, by way of example, a block diagram of an embodiment of a system 1800 (e.g., a computing system) for performing analysis of autonomic nervous system condition data (e.g., by the SCS surgery planning system 1242, planning model 1246, SCS treatment planning system 1244) in connection with the data processing operations discussed above. The system 1800 may be integrated with or coupled to a computing device, a remote control device, patient programmer device, clinician programmer device, program modeling system, or other external device, associated with neurostimulation planning or treatment. In some examples, the system 1800 may be a networked device (server) connected via a network (or combination of networks) which communicates to one or more devices (clients) using a communication interface 1808 (e.g., communication hardware which implements software network interfaces and services). The network may include local, short-range, or long-range networks, such as Bluetooth, cellular, IEEE 802.11 (Wi-Fi), or other wired or wireless networks.

The system 1800 includes a processor 1802 and a memory 1804, which can be optionally included as part of data processing circuitry 1806. The processor 1802 may be any single processor or group of processors that act cooperatively. The memory 1804 may be any type of memory, including volatile or non-volatile memory. The memory 1804 may include instructions, which when executed by the processor 1802, cause the processor 1802 to implement data processing, a user interface 1810, or to enable other features of the data processing circuitry 1806. Thus, electronic operations in the system 1800 may be performed by the processor 1802 or the circuitry 1806.

For example, the processor 1802 or circuitry 1806 may implement any of the features of the method 1700 to obtain and process user input, to determine severity scores associated with characteristics of an autonomic condition, to determine neurostimulation programming settings based on the severity scores, and to output or provide graphical representations and displays based on the severity scores, neural targeting locations, or stimulation settings. It will be understood that the processor 1802 or circuitry 1806 may also implement aspects of the logic and processing described above, for use in various forms of device programming or related device actions.

FIG. 19 illustrates, by way of example, a block diagram of an embodiment of a system 1900 (e.g., a computing system) implementing neurostimulation programming circuitry 1906 to cause programming of an implantable electrical neurostimulation device, for accomplishing the autonomic nervous system treatment objectives in a human subject based on neurostimulation programming as discussed herein. The system 1900 may be operated by a clinician, a patient, a caregiver, a medical facility, a research institution, a medical device manufacturer or distributor, and embodied in a number of different computing platforms. The system 1900 may be a remote control device, patient programmer device, program modeling system, or other external device, including a regulated device used to directly implement programming commands and modification with a neurostimulation device. In some examples, the system 1900 may be a networked device connected via a network (or combination of networks) to a computing system operating a user interface computing system using a communication interface 1908. The network may include local, short-range, or long-range networks, such as Bluetooth, cellular, IEEE 802.11 (Wi-Fi), or other wired or wireless networks.

The system 1900 includes a processor 1902 and a memory 1904, which can be optionally included as part of neurostimulation programming circuitry 1906. The processor 1902 may be any single processor or group of processors that act cooperatively. The memory 1904 may be any type of memory, including volatile or non-volatile memory. The memory 1904 may include instructions, which when executed by the processor 1902, cause the processor 1902 to implement the features of the neurostimulation programming circuitry 1906. Thus, the electronic operations in the system 1900 may be performed by the processor 1902 or the circuitry 1906.

The processor 1902 or circuitry 1906 may directly or indirectly implement neurostimulation operations associated with the method 1700, including the use of neurostimulation device programming based on patient-specific surgical planning or programming. The processor 1902 or circuitry 1906 may further provide data and commands to assist the processing and implementation of the programming using communication interface 1908 or a neurostimulation device interface 1910. It will be understood that the processor 1902 or circuitry 1906 may also implement other aspects of the device data processing or device programming functionality described above.

Various circuits or circuitry may, alone or in combination, perform or implement the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, etc.) to encode instructions of the specific operation. In an example, the instructions enable embedded hardware (e.g., the execution units or a loading mechanism) to create members of the circuitry in hardware via variable connections to carry out portions of the specific operation when in operation. Accordingly, the computer readable medium may be communicatively coupled to the other components of the circuitry when the device is operating. In specific examples, functions of the data processing circuitry 1806 or the neurostimulation programming circuitry 1906 may be implemented as a part of a microprocessor circuit. The microprocessor circuit can be a dedicated processor such as a digital signal processor, application specific integrated circuit (ASIC), microprocessor, or other type of processor for processing information. Alternatively, the microprocessor circuit can be a general purpose processor that can receive and execute a set of instructions of performing the methods or techniques described herein.

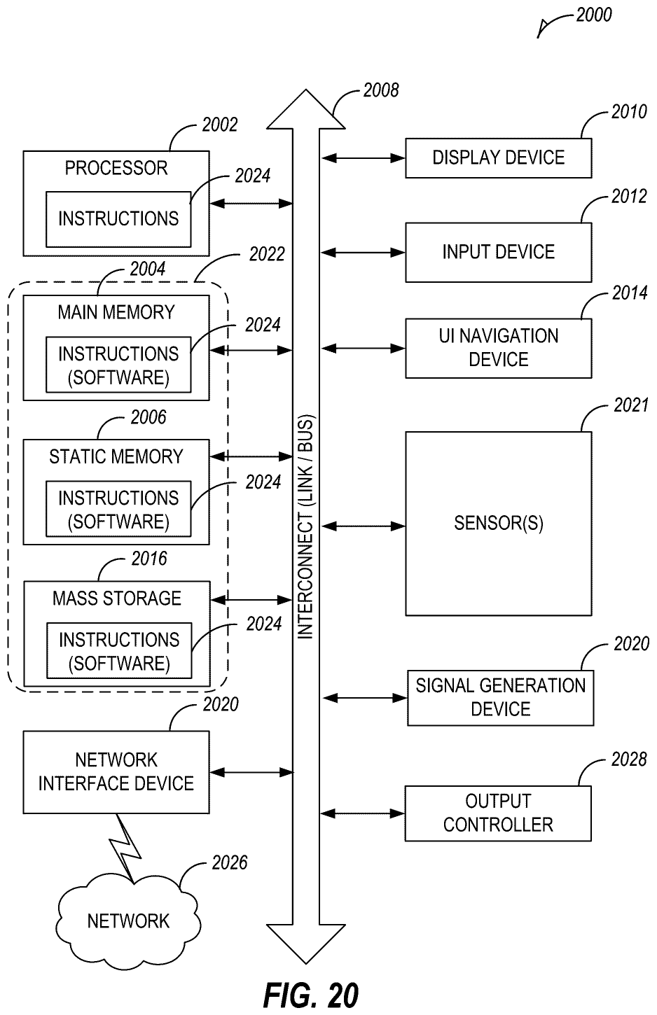

FIG. 20 illustrates generally a block diagram of an example machine 2000 upon which any one or more of the techniques (e.g., methodologies) discussed herein may perform. Portions of this description may apply to the computing framework of various portions of the neuromodulation device or the external programming device.

In alternative embodiments, the machine 2000 may operate as a standalone device or may be connected (e.g., networked) to other machines. In a networked deployment, the machine 2000 may operate in the capacity of a server machine, a client machine, or both in server-client network environments. In an example, the machine 2000 may act as a peer machine in peer-to-peer (P2P) (or other distributed) network environment. The machine 2000 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 2000 may include a hardware processor 2002 (e.g., a central processing unit (CPU), a graphics processing unit (GPU), a hardware processor core, or any combination thereof), a main memory 2004 and a static memory 2006, some or all of which may communicate with each other via an interlink (e.g., bus) 2008. The machine 2000 may further include a display unit 2010 (e.g., a raster display, vector display, holographic display, etc.), an alphanumeric input device 2012 (e.g., a keyboard), and a user interface (UI) navigation device 2014 (e.g., a mouse). In an example, the display unit 2010, input device 2012 and UI navigation device 2014 may be a touch screen display. The machine 2000 may additionally include a storage device (e.g., drive unit) 2016, a signal generation device 2018 (e.g., a speaker), a network interface device 2020, and one or more sensors 2021, such as a global positioning system (GPS) sensor, compass, accelerometer, or other sensors. The machine 2000 may include an output controller 2028, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 2016 may provide a machine readable medium 2022 on which is stored one or more sets of data structures or instructions 2024 (e.g., software) embodying or utilized by any one or more of the techniques or functions described herein. The instructions 2024 may also reside, completely or at least partially, within the main memory 2004, within static memory 2006, or within the hardware processor 2002 during execution thereof by the machine 2000. In an example, one or any combination of the hardware processor 2002, the main memory 2004, the static memory 2006, or the storage device 2016 may constitute machine readable media.

While the machine-readable medium 2022 is illustrated as a single medium, the term “machine readable medium” may include a single medium or multiple media (e.g., a centralized or distributed database, and/or associated caches and servers) configured to store the one or more instructions 2024.

The term “machine readable medium” may include any medium that is capable of storing, encoding, or carrying instructions for execution by the machine 2000 and that cause the machine 2000 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

The instructions 2024 may further be transmitted or received over a communication network 2026 using a transmission medium via the network interface device 2020 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, 3GPP cellular data networks provided according to 4G, 5G, or 6G standards), IEEE 802.15.4 family of standards, peer-to-peer (P2P) networks, among others. In an example, the network interface device 2020 may include one or more physical jacks (e.g., Ethernet, coaxial, or phone jacks) or one or more antennas to connect to the communication network 2026. In an example, the network interface device 2020 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 2000, and includes digital or analog communications signals or other intangible medium to facilitate communication of such software.

Various embodiments are illustrated in the figures above. One or more features from one or more of these embodiments may be combined to form other embodiments.

The method examples described herein may be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code may form portions of computer program products. Further, the code may be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A data processing system for planning neurostimulation programming of a patient, comprising:
at least one memory device configured to store patient data corresponding to multiple characteristics of an autonomic condition of the patient;
at least one processor configured to:
identify neural targets of the patient corresponding to respective leads and electrodes of a neurostimulation system;
determine, based on the patient data, respective severity scores corresponding to the multiple characteristics of the autonomic condition;
generate, using the respective severity scores, neurostimulation programming settings for use at the neural targets; and
output programming data, based on the neurostimulation programming settings, for controlling neurostimulation with the neurostimulation system.

2. The data processing system of claim 1, wherein the neural targets of the patient include spinal neural targets, and wherein the neurostimulation includes spinal cord stimulation (SCS) to be provided via the respective leads and electrodes to the spinal neural targets.

3. The data processing system of claim 1, wherein the data processing system is further configured to:
communicate with a user interface device to output the respective severity scores in a graphical user interface, wherein the user interface device is configured to output a visualization of the respective severity scores that depicts relative severity for each of the multiple characteristics of the autonomic condition.

4. The data processing system of claim 3, wherein the respective severity scores are represented in the graphical user interface within a defined range or groups of ranges for each of the multiple characteristics of the autonomic condition.

5. The data processing system of claim 3, wherein the visualization of the respective severity scores comprises a circular graph that positions the multiple characteristics of the autonomic condition at respective locations in the circular graph,
wherein the circular graph includes an overlay area that maps the respective severity scores onto the respective locations in the circular graph, and
wherein the circular graph is configured to receive user interaction to change the respective severity scores at the respective locations in the circular graph.

6. The data processing system of claim 1, wherein the data processing system is further configured to:
communicate with a user interface device to represent the multiple characteristics of an autonomic condition of the patient, wherein the user interface device is further configured to output a graphical user interface including at least one of:
an anatomical visualization including at least one visceral map or dermatomal map, the anatomical visualization corresponding to symptoms or treatment effects at the neural targets; or
a heat map corresponding to symptoms or treatment effects at the neural targets, and wherein the heat map is overlaid on the neural targets on a visualization of a spinal cord for the patient.

7. The data processing system of claim 1, wherein the respective severity scores are determined based on the patient data received in a symptom questionnaire, and
wherein the symptom questionnaire provides a numerical measurement of effects of the autonomic condition in respective anatomical systems or organs that correspond to the multiple characteristics of the autonomic condition.

8. The data processing system of claim 1, wherein the neurostimulation programming settings are determined based on a mapping of the neural targets to a severity score value corresponding to each of the multiple characteristics of the autonomic condition, and
wherein the mapping of the neural targets is based on a score computed from a likelihood of targeting a respective characteristic of the autonomic condition and a severity weight for the respective characteristic of the autonomic condition.

9. The data processing system of claim 1, wherein the neurostimulation programming settings includes information for: one or more spatial targets, one or more frequencies, and one or more pulse-widths used for the neurostimulation; and
wherein the neurostimulation programming settings are generated based on a configuration of the respective leads and electrodes of the neurostimulation system used for the patient, wherein the configuration provides specifications for: a type of implantable pulse generator, a type of lead, a number of leads, and a number of electrodes on respective leads.

10. The data processing system of claim 1, further comprising:
communication circuitry configured to provide programming instructions for an electrostimulator of the neurostimulation system, the programming instructions corresponding to the neurostimulation programming settings to control stimulation of at respective neural targets of the patient via the respective leads and electrodes;
wherein the programming instructions includes one or more stimulation parameters including:
an electrode configuration;
one or more stimulation pulse parameters including a pulse amplitude, a pulse width, or a stimulation frequency;
a stimulation pulse waveform;
an ON-OFF cycling scheme comprising an ON period for delivering stimulation pulses and a subsequent stimulation-free OFF period; or
a charge per second (CPS) or a charge per hour (CPH) delivered to a respective neural target.

11. A method for planning neurostimulation programming of a patient, comprising:
receiving patient data corresponding to multiple characteristics of an autonomic condition of the patient;
identifying neural targets of the patient corresponding to respective leads and electrodes of a neurostimulation system;
determining, based on the patient data, respective severity scores corresponding to the multiple characteristics of the autonomic condition;
generating, using the respective severity scores, neurostimulation programming settings for use at the neural targets; and
outputting programming data, based on the neurostimulation programming settings, for controlling neurostimulation with the neurostimulation system.

12. The method of claim 11, wherein the neural targets of the patient include spinal neural targets, and wherein the neurostimulation includes spinal cord stimulation (SCS) to be provided via the respective leads and electrodes to the spinal neural targets.

13. The method of claim 11, further comprising:
communicating with a user interface device to output the respective severity scores in a graphical user interface, wherein the user interface device is configured to output a visualization of the respective severity scores that depicts relative severity for each of the multiple characteristics of the autonomic condition.

14. The method of claim 13, wherein the respective severity scores are represented in the graphical user interface within a defined range or groups of ranges for each of the multiple characteristics of the autonomic condition.

15. The method of claim 13, wherein the visualization of the respective severity scores comprises a circular graph that positions the multiple characteristics of the autonomic condition at respective locations in the circular graph,
wherein the circular graph includes an overlay area that maps the respective severity scores onto the respective locations in the circular graph, and
wherein the circular graph is configured to receive user interaction to change the respective severity scores at the respective locations in the circular graph.

16. The method of claim 11, further comprising:
communicating with a user interface device to represent the multiple characteristics of an autonomic condition of the patient, wherein the user interface device is further configured to output a graphical user interface including at least one of:
an anatomical visualization including at least one visceral map or dermatomal map, the anatomical visualization corresponding to symptoms or treatment effects at the neural targets; or
a heat map corresponding to symptoms or treatment effects at the neural targets, and wherein the heat map is overlaid on the neural targets on a visualization of a spinal cord for the patient.

17. The method of claim 11, wherein the respective severity scores are determined based on the patient data received in a symptom questionnaire, and
wherein the symptom questionnaire provides a numerical measurement of effects of the autonomic condition in respective anatomical systems or organs that correspond to the multiple characteristics of the autonomic condition.

18. The method of claim 11, wherein the neurostimulation programming settings are determined based on a mapping of the neural targets to a severity score value corresponding to each of the multiple characteristics of the autonomic condition, and
wherein the mapping of the neural targets is based on a score computed from a likelihood of targeting a respective characteristic of the autonomic condition and a severity weight for the respective characteristic of the autonomic condition.

19. The method of claim 11, wherein the neurostimulation programming settings includes information for: one or more spatial targets, one or more frequencies, and one or more pulse-widths used for the neurostimulation; and
wherein the neurostimulation programming settings are generated based on a configuration of the respective leads and electrodes of the neurostimulation system used for the patient, wherein the configuration provides specifications for: a type of implantable pulse generator, a type of lead, a number of leads, and a number of electrodes on respective leads.

20. The method of claim 11, further comprising:
communicating programming instructions for an electrostimulator of the neurostimulation system, the programming instructions corresponding to the neurostimulation programming settings to control stimulation of at respective neural targets of the patient via the respective leads and electrodes;
wherein the programming instructions includes one or more stimulation parameters including:
an electrode configuration;
one or more stimulation pulse parameters including a pulse amplitude, a pulse width, or a stimulation frequency;
a stimulation pulse waveform;
an ON-OFF cycling scheme comprising an ON period for delivering stimulation pulses and a subsequent stimulation-free OFF period; or
a charge per second (CPS) or a charge per hour (CPH) delivered to a respective neural target.
